(19) United States
(12) Patent Application Publication
(10) Pub. No.: US 2022/0125829 A1
(43) Pub. Date: Apr. 28, 2022

(54) METHODS AND COMPOSITIONS FOR TREATING MEIBOMIAN GLAND DYSFUNCTION, DRY EYE DISEASE, AND RELATED DISORDERS

(71) Applicant: Hovione Scientia Limited, Cork (IE)

(72) Inventors: Carla Maria dos Santos Vozone, East Windsor, NJ (US); Mohammad Salman, East Windsor, NJ (US); George Nathaniel Magrath, III, Mount Pleasant, SC (US); Courtney Rouse Smith, Gainesville, GA (US)

(21) Appl. No.: 17/511,936

(22) Filed: Oct. 27, 2021

Related U.S. Application Data
(60) Provisional application No. 63/106,672, filed on Oct. 28, 2020.

(51) Int. Cl. A61K 31/745; A61K 9/00; A61K 33/00; A61P 27/04

(52) U.S. Cl. CPC A61K 31/745; A61P 27/04; A61K 33/00; A61K 9/0048

(57) ABSTRACT

The invention provides methods, compositions, and kits containing a pharmaceutical composition, for treating meibomian gland dysfunction, dry eye disease, and related disorders.

### CROSS-REFERENCE TO RELATED APPLICATION

This application claims the benefit of and priority to U.S. Provisional Patent Application Ser. No. 63/106,672, filed Oct. 28, 2020, the contents of which are hereby incorporated by reference in their entirety.

### FIELD OF THE INVENTION

The invention provides methods, compositions, and kits containing a pharmaceutical composition, for treating meibomian gland dysfunction, dry eye disease, and related disorders.

### BACKGROUND

Functioning meibomian glands play a critical role in maintaining optimal ocular surface conditions. One common disorder observed in patients by eye care professionals, including ophthalmologists and optometrists, is meibomian gland dysfunction. Meibomian gland dysfunction typically features meibomian glands that either hyposecrete or are obstructed. Meibomian gland dysfunction has historically been treated on a chronic basis through either mechanical therapy (e.g., eyelid hygiene, eyelid massage, or eyelid compression/expression) alone or in combination with topical or systemic antibiotics or topical immunosuppressants such as steroids or cyclosporine. The need exists for meibomian gland dysfunction therapies that are more effective and/or have reduced adverse side effects.

Dry Eye Disease (DED) is a relatively common condition characterized by inadequate tear film protection of the cornea. DED often causes ocular discomfort, a degradation in visual performance (reading speed, contrast sensitivity), and a loss of productivity. Many millions of people worldwide suffer from DED, with it being more frequently diagnosed in aging patient populations. Currently available options for treating DED are limited and costly.

The present invention addresses the need for improved therapies for meibomian gland dysfunction, dry eye disease, and related disorders and provides other related advantages.

### SUMMARY

The invention provides methods, compositions, and kits containing a pharmaceutical composition, for treating meibomian gland dysfunction, dry eye disease, and related disorders. The pharmaceutical composition contains (i) an ethylene-propylene-styrene copolymer and (ii) optionally one or more of a butylene-ethylene-styrene copolymer, mineral oil, an antioxidant and one or more pharmaceutically acceptable carriers and/or excipients. Preferably, the pharmaceutical composition contains an ethylene-propylene-styrene copolymer, a butylene-ethylene-styrene copolymer, mineral oil, and an antioxidant. The pharmaceutical composition is preferably in the form of a gel and is topically administered to the eyelid margin of the patient's eye. Without limiting the scope of patients that achieve benefits from the therapy, it has been observed that patients having non-inflammatory meibomian gland dysfunction respond particularly well to treatment using the pharmaceutical composition administered twice per day. Exemplary aspects and embodiments of the invention are described below.

One aspect of the invention provides a method of treating meibomian gland dysfunction. The method consists of topically administering to the eyelid margin of a patient in need thereof a therapeutically effective amount of a pharmaceutical composition to treat the meibomian gland dysfunction, wherein the pharmaceutical composition consists of: (a) an ethylene-propylene-styrene copolymer; (b) optionally a butylene-ethylene-styrene copolymer; (c) optionally mineral oil; (d) optionally an antioxidant; and (e) optionally one or more pharmaceutically acceptable carriers and/or excipients. Additional pharmaceutical compositions for use in the method are described in the detailed description, along with additional features and benefits of the method.

Another aspect of the invention provides a method of treating dry eye disease. The method consists of topically administering to the eyelid margin of a patient in need thereof a therapeutically effective amount of a pharmaceutical composition to treat the dry eye disease, wherein the pharmaceutical composition consists of: (a) an ethylene-propylene-styrene copolymer; (b) optionally a butylene-ethylene-styrene copolymer; (c) optionally mineral oil; (d) optionally an antioxidant; and (e) optionally one or more pharmaceutically acceptable carriers and/or excipients. Additional pharmaceutical compositions for use in the method are described in the detailed description, along with additional features and benefits of the method.

The above methods may be further characterized according to, for example, features of the pharmaceutical composition, patients to receive treatment, the dose of the pharmaceutical composition, the frequency of administration of the pharmaceutical composition, and results produced by the method. In certain embodiments, the patient has evaporative dry eye disease. In certain embodiments, an amount of from about 45 μL to about 55 μL of the pharmaceutical composition is topically administered to the eyelid margin of the patient per administration. In certain embodiments, the pharmaceutical composition is administered twice per day. In certain embodiments, the pharmaceutical composition is administered up to twice per day as needed. These and other features are described in more detail herein below.

Also provided is a pharmaceutical composition for use in treating medical conditions described herein. Such use may employ embodiments described herein for the therapeutic methods, such as the features of the pharmaceutical composition, patients to receive treatment, the frequency of administration of the pharmaceutical composition, and results produced by the use.

### BRIEF DESCRIPTION OF FIGURES

FIG. 1 depicts a diagram illustrating regions of a patient's eye assessed using Fluorescein Corneal Staining to determine corneal damage.

FIG. 2 is a graph showing results of a viscosity vs. shear rate analysis for the polymeric hydrocarbon gelling agent sold commercially under the tradename VERSAGEL® M-750, as further described herein below. Different lines on the graph correspond to different lots of polymeric hydrocarbon gelling agent and/or a repetition of the viscosity vs. shear rate analysis. Shear rate sweep testing was performed on a research rheometer (DHR2, TA Instruments) fitted with a 40 mm crosshatched plate measuring system. Following a 30 second equilibration time at 25° C., the samples were exposed to a 30 second pre-shear at a rate of 0.1 s⁻¹ followed immediately by a shear rate sweep, 0.1 s⁻¹ to 10,000 s⁻¹, logarithmically scaled, 6 points per decade of shear rate, shear applied for 30 seconds at each rate with viscosity calculated over the final 5 seconds of each step.

FIG. 3 is a graph showing results of a yield stress analysis for the polymeric hydrocarbon gelling agent sold commercially under the tradename VERSAGEL® M-750, as further described herein below. Different lines on the graph correspond to different lots of polymeric hydrocarbon gelling agent and/or a repetition of the yield stress analysis. Oscillation testing was performed on a research rheometer (DHR2, TA Instruments) fitted with a 40 mm 1° cone and plate measuring system. Following a 60 second equilibration time at 25° C., the samples were exposed to an oscillatory stress sweep ranging from 0.1 Pa, logarithmically scaled, 10 points per decade of stress, 1 Hz oscillation frequency. A step termination was set such that if at any point the oscillation strain exceeded 1500% the test would terminate.

FIG. 4 is a graph showing results of a normal stress analysis for the polymeric hydrocarbon gelling agent sold commercially under the tradename VERSAGEL® M-750, as further described herein below. Different lines on the graph correspond to different lots of polymeric hydrocarbon gelling agent and/or a repetition of the normal stress analysis. Shear rate sweep testing was performed on a research rheometer (DHR2, TA Instruments) fitted with a 40 mm crosshatched plate measuring system. Normal stress data was quantified during the shear sweep testing, as described above in connection with FIG. 2.

FIG. 5 is a line graph depicting VAS discomfort (mean change from baseline) results from the clinical study in Example 2.

FIG. 6 is a line graph depicting VAS eye dryness (mean change from baseline) results from the clinical study in Example 2.

FIG. 7 is a line graph depicting VAS foreign body sensation (mean change from baseline) results from the clinical study in Example 2.

FIG. 8 is a line graph depicting vascular engorgement (mean change from baseline) results from the clinical study in Example 2.

FIG. 9 is a line graph depicting VAS discomfort (mean change from baseline) results observed in the MMP-9-Negative Subgroup of subjects from the clinical study in Example 2.

### DETAILED DESCRIPTION OF THE INVENTION

The invention provides methods, compositions, and kits containing a pharmaceutical composition, for treating meibomian gland dysfunction, dry eye disease, and related disorders. The pharmaceutical composition contains (i) an ethylene-propylene-styrene copolymer and (ii) optionally one or more of a butylene-ethylene-styrene copolymer, mineral oil, an antioxidant and one or more pharmaceutically acceptable carriers and/or excipients. Preferably, the pharmaceutical composition contains an ethylene-propylene-styrene copolymer, a butylene-ethylene-styrene copolymer, mineral oil, and an antioxidant. The pharmaceutical composition is preferably in the form of a gel and is topically administered to the eyelid margin of the patient's eye. Without limiting the scope of patients that achieve benefits from the therapy, it has been observed that patients having non-inflammatory meibomian gland dysfunction respond particularly well to treatment using the pharmaceutical composition administered twice per day. Various aspects of the invention are set forth below in sections; however, aspects of the invention described in one particular section are not to be limited to any particular section.

### Definitions

To facilitate an understanding of the present invention, a number of terms and phrases are defined below.

The terms “a,” “an” and “the” as used herein mean “one or more” and include the plural unless the context is inappropriate.

As used herein, the term “non-inflammatory meibomian gland dysfunction” refers to meibomian gland dysfunction where there is no substantial amount of inflammation in the patient's meibomian glands. Non-inflammatory meibomian gland dysfunction can be assessed, for example, by measuring the concentration of matrix metalloproteinase-9 (MMP-9) in the patient's tear film. A MMP-9 tear film concentration less than about 35 ng/mL is generally indicative of no substantial amount of inflammation in the patient's meibomian glands. More preferably, a MMP-9 tear film concentration less than about 30, 25, 20, 15, 10, or 5 ng/mL is generally indicative of no substantial amount of inflammation in the patient's meibomian glands. Visual indicators of inflammation of the meibomian glands may also be used to determine if the patient has a substantial amount of inflammation in their meibomian glands.

As used herein, the term “patient” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results. Unless specified otherwise, an effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutically acceptable carrier” refers to any of the standard insert pharmaceutical carriers. Exemplary pharmaceutically acceptable carriers include, for example, water, isotonic saline, phosphate buffered saline solution, cellulose, Ringer's solution, corn starch, potato starch, lactose, glycose, and sucrose. For examples of carriers, see Martin in Remington's Pharmaceutical Sciences, 15th Ed., Mack Publ. Co., Easton, Pa. [1975].

As used herein, the term “pharmaceutically acceptable excipient” refers to any of the standard inert pharmaceutical excipients included in a pharmaceutical dosage form to, for example, aid in manufacturing, protect the active ingredient(s), and/or enhance stability of the pharmaceutical dosage form. Exemplary pharmaceutically acceptable excipients include, for example, aluminium hydroxide, aluminum potassium sulfate dodecahydrate, calcium phosphate, aluminum phosphate, sodium acetate, acetic acid, citric acid, sodium citrate, malic acid, sodium maleate, magnesium chloride hexahydrate, potassium metaphosphate, ammonium sulfate, potassium dihydrogen phosphate, dipotassium hydrogen phosphate, calcium hydrogen phosphate, sodium sulfite, sodium sulfate, sodium chloride, talc, and silicon dioxide.

As used herein, the term “pharmaceutically acceptable salt” refers to any pharmaceutically acceptable salt (e.g., acid or base) of a compound of the present invention which, upon administration to a subject, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate (mesylate), 2-naphthalenesulfonate, nicotinate, oxalate, palmoate, pectinate, persulfate, phenylpropionate, picrate, pivalate, propionate, succinate, sulfate, tartrate, thiocyanate, tosylate, undecanoate, and the like. Other examples of salts include anions of the compounds of the present invention compounded with a suitable cation such as Nat, NH₄+, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Therapeutic Methods

The invention provides methods for treating patients suffering from meibomian gland dysfunction, dry eye disease, and related disorders by administering a pharmaceutical composition to the patient. The pharmaceutical composition contains (i) an ethylene-propylene-styrene copolymer and (ii) optionally one or more of a butylene-ethylene-styrene copolymer, mineral oil, an antioxidant and one or more pharmaceutically acceptable carriers and/or excipients. Various aspects and embodiments of the therapeutic methods are described in the sections below. The sections are arranged for convenience and information in one section is not to be limited to that section, but may be applied to methods in other sections.

### A. First Method

One aspect of the invention provides a method of treating meibomian gland dysfunction. The method consists of topically administering to the eyelid margin of a patient in need thereof a therapeutically effective amount of a pharmaceutical composition to treat the meibomian gland dysfunction, wherein the pharmaceutical composition consists of:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

Additional exemplary features that may characterize the method are provided below and include, for example, features of the pharmaceutical composition, patients to receive treatment, the dosing regimen used to administer the pharmaceutical composition to the patient, and results produced by the method. A more thorough description of such features is provided below.

### B. Second Method

Another aspect of the invention provides a method of treating meibomian gland dysfunction. The method comprises topically administering to the eyelid margin of a patient in need thereof a therapeutically effective amount of a pharmaceutical composition to treat the meibomian gland dysfunction, wherein the pharmaceutical composition consists of:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

Additional exemplary features that may characterize the method are provided below and include, for example, features of the pharmaceutical composition, patients to receive treatment, the dosing regimen used to administer the pharmaceutical composition to the patient, and results produced by the method. A more thorough description of such features is provided below.

### C. Third Method

Another aspect of the invention provides a method of treating meibomian gland dysfunction. The method comprises topically administering to the eyelid margin of a patient in need thereof a therapeutically effective amount of a pharmaceutical composition to treat the meibomian gland dysfunction, wherein the pharmaceutical composition comprises:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

Additional exemplary features that may characterize the method are provided below and include, for example, features of the pharmaceutical composition, patients to receive treatment, the dosing regimen used to administer the pharmaceutical composition to the patient, and results produced by the method. A more thorough description of such features is provided below.

### D. Fourth Method

Another aspect of the invention provides a method of treating meibomian gland dysfunction. The method comprises topically administering to the eyelid margin of a patient in need thereof a therapeutically effective amount of a single therapeutic agent to treat the meibomian gland dysfunction, wherein the single therapeutic agent is the only agent administered to the patient that treats meibomian gland dysfunction, and the single therapeutic agent consists of:
(a) an ethylene-propylene-styrene copolymer;
(b) optionally a butylene-ethylene-styrene copolymer; and
(c) optionally mineral oil.

In certain embodiments, the single therapeutic agent is formulated into a pharmaceutical composition for administration to the patient.

Additional exemplary features that may characterize the method are provided below and include, for example, features of the single therapeutic agent, patients to receive treatment, the dosing regimen used to administer the single therapeutic agent to the patient, and results produced by the method. A more thorough description of such features is provided below.

### E. Fifth Method

Another aspect of the invention provides a method of treating dry eye disease. The method consists of topically administering to the eyelid margin of a patient in need thereof a therapeutically effective amount of a pharmaceutical composition to treat the dry eye disease, wherein the pharmaceutical composition consists of:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

The method may be further characterized according to the type of dry eye disease. For example, in certain embodiments, the dry eye disease is aqueous tear deficiency dry eye disease. In certain embodiments, the dry eye disease is evaporative dry eye disease.

Additional exemplary features that may characterize the method are provided below and include, for example, features of the pharmaceutical composition, patients to receive treatment, the dosing regimen used to administer the pharmaceutical composition to the patient, and results produced by the method. A more thorough description of such features is provided below.

### F. Sixth Method

Another aspect of the invention provides a method of treating dry eye disease. The method comprises topically administering to the eyelid margin of a patient in need thereof a therapeutically effective amount of a pharmaceutical composition to treat the dry eye disease, wherein the pharmaceutical composition consists of:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

The method may be further characterized according to the type of dry eye disease. For example, in certain embodiments, the dry eye disease is aqueous tear deficiency dry eye disease. In certain embodiments, the dry eye disease is evaporative dry eye disease.

Additional exemplary features that may characterize the method are provided below and include, for example, features of the pharmaceutical composition, patients to receive treatment, the dosing regimen used to administer the pharmaceutical composition to the patient, and results produced by the method. A more thorough description of such features is provided below.

### G. Seventh Method

Another aspect of the invention provides a method of treating dry eye disease. The method comprises topically administering to the eyelid margin of a patient in need thereof a therapeutically effective amount of a pharmaceutical composition to treat the dry eye disease, wherein the pharmaceutical composition comprises:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

The method may be further characterized according to the type of dry eye disease. For example, in certain embodiments, the dry eye disease is aqueous tear deficiency dry eye disease. In certain embodiments, the dry eye disease is evaporative dry eye disease.

Additional exemplary features that may characterize the method are provided below and include, for example, features of the pharmaceutical composition, patients to receive treatment, the dosing regimen used to administer the pharmaceutical composition to the patient, and results produced by the method. A more thorough description of such features is provided below.

### H. Eighth Method

Another aspect of the invention provides a method of treating dry eye disease. The method comprises topically administering to the eyelid margin of a patient in need thereof a therapeutically effective amount of a single therapeutic agent to treat the dry eye disease, wherein the single therapeutic agent is the only agent administered to the patient that treats dry eye disease, and the single therapeutic agent consists of:
(a) an ethylene-propylene-styrene copolymer;
(b) optionally a butylene-ethylene-styrene copolymer; and
(c) optionally mineral oil.

The method may be further characterized according to the type of dry eye disease. For example, in certain embodiments, the dry eye disease is aqueous tear deficiency dry eye disease. In certain embodiments, the dry eye disease is evaporative dry eye disease.

Additional exemplary features that may characterize the method are provided below and include, for example, features of the single therapeutic agent, patients to receive treatment, the dosing regimen used to administer the single therapeutic agent to the patient, and results produced by the method. A more thorough description of such features is provided below.

### I. Nineth Method

Another aspect of the invention provides a method of treating a disorder selected from the group consisting of blepharitis and meibomitis. The method comprises topically administering to the eyelid margin of a patient in need thereof a therapeutically effective amount of a pharmaceutical composition to treat the disorder, wherein the pharmaceutical composition comprises:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

In certain embodiments, the invention provides a method of treating a disorder selected from the group consisting of blepharitis and meibomitis, wherein the method comprises topically administering to the eyelid margin of a patient in need thereof a therapeutically effective amount of a pharmaceutical composition to treat the disorder, wherein the pharmaceutical composition consists of:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

In certain embodiments, the invention provides a method of treating a disorder selected from the group consisting of blepharitis and meibomitis, wherein the method consists of topically administering to the eyelid margin of a patient in need thereof a therapeutically effective amount of a pharmaceutical composition to treat the disorder, wherein the pharmaceutical composition consists of:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

The method may be further characterized according to the type of disorder. For example, in certain embodiments, the disorder is blepharitis. In certain embodiments, the disorder is meibomitis. Additional exemplary features that may characterize the method are provided below and include, for example, features of the pharmaceutical composition, patients to receive treatment, the dosing regimen used to administer the pharmaceutical composition to the patient, and results produced by the method. A more thorough description of such features is provided below.

### J. Tenth Method

Another aspect of the invention provides a method of treating disorder selected from the group consisting of blepharitis and meibomitis. The method comprises topically administering to the eyelid margin of a patient in need thereof a therapeutically effective amount of a single therapeutic agent to treat the disorder, wherein the single therapeutic agent is the only agent administered to the patient that treats the disorder, and the single therapeutic agent consists of:
(a) an ethylene-propylene-styrene copolymer;
(b) optionally a butylene-ethylene-styrene copolymer; and
(c) optionally mineral oil.

The method may be further characterized according to the type of disorder. For example, in certain embodiments, the disorder is blepharitis. In certain embodiments, the disorder is meibomitis.

In certain embodiments, the single therapeutic agent is formulated into a pharmaceutical composition for administration to the patient.

Additional exemplary features that may characterize the method are provided below and include, for example, features of the single therapeutic agent, patients to receive treatment, the dosing regimen used to administer the single therapeutic agent to the patient, and results produced by the method. A more thorough description of such features is provided below.

### K. Eleventh Method

Another aspect of the invention provides a method of treating a disorder selected from the group consisting of meibomian gland dysfunction and dry eye disease. The method consists of topically administering to the eyelid margin of a patient in need thereof a therapeutically effective amount of a pharmaceutical composition to treat the disorder, wherein the pharmaceutical composition consists of:
(a) mineral oil;
(b) a gelling agent; and
(c) optionally one or more pharmaceutically acceptable carriers and/or excipients.

The method may be further characterized according to the type of disorder. For example, in certain embodiments, the disorder is meibomian gland dysfunction. In certain embodiments, the disorder is dry eye disease.

Additional exemplary features that may characterize the method are provided below and include, for example, features of the pharmaceutical composition and results produced by the method. A more thorough description of such features is provided below.

In certain embodiments, the mineral oil is present in the pharmaceutical composition in an amount of at least 90% (w/w) of the pharmaceutical composition. In certain embodiments, the mineral oil is present in the pharmaceutical composition in an amount of at least 95% (w/w) of the pharmaceutical composition.

### L. Additional Exemplary Features of the First, Second, Third, and Fourth Therapeutic Methods

Additional exemplary features that may characterize the First, Second, Third, and Fourth Therapeutic Methods described herein are provided below and include, for example, characteristics of meibomian gland dysfunction, patients to be treated, and results produced by the methods. A more thorough description of such features is provided below. The invention embraces all permutations and combinations of these features.

### Type of Meibomian Gland Dysfunction

The methods may be further characterized according to the type of meibomian gland dysfunction. For example, in certain embodiments, the meibomian gland dysfunction is mild meibomian gland dysfunction. In certain embodiments, the meibomian gland dysfunction is moderate meibomian gland dysfunction. In certain embodiments, the meibomian gland dysfunction is non-inflammatory meibomian gland dysfunction. In certain embodiments, the patient has an inflamed meibomian gland.

### Additional Conditions Suffered by the Patient

The methods may be further characterized according to additional conditions suffered by the patient. For example, in certain embodiments, the patient also suffers from dry eye disease. In certain embodiments, the patient also suffers from evaporative dry eye disease. In certain embodiments, the patient also suffers from demodex mites on at least one of their eyelids.

### Meibomian Gland Dysfunction Symptom Flare Frequency

The methods may be further characterized according to the frequency of symptom flare due to meibomian gland dysfunction in the patient to be treated. For example, in certain embodiments, the patient experiences a symptom flare due to meibomian gland dysfunction on an average of at least 1, 2, 3, 4, 5, 6, 7, 8, 9, 10, 15, 20, 25, or 30 times per day. In certain embodiments, the patient experiences a symptom flare due to meibomian gland dysfunction on an average of at least one time per day. In certain embodiments, the patient experiences a symptom flare due to meibomian gland dysfunction on an average of at least 2 times per day. In certain embodiments, the patient experiences a symptom flare due to meibomian gland dysfunction on an average of at least 3 times per day. In certain embodiments, the patient experiences a symptom flare due to meibomian gland dysfunction on an average of at least one time per every two days. In certain embodiments, the patient experiences a symptom flare due to meibomian gland dysfunction on an average of from once per day to 5 times per day.

In certain embodiments, the patient experiences a symptom flare due to meibomian gland dysfunction on an average of at least 1, 2, 3, 4, 5, 6, 7, 8, 9, 10, 15, 20, 25, or 30 times per week. In certain embodiments, the patient experiences a symptom flare due to meibomian gland dysfunction on an average of at least 5 times per week. In certain embodiments, the patient experiences a symptom flare due to meibomian gland dysfunction on an average of at least 10 times per week. In certain embodiments, the patient experiences a symptom flare due to meibomian gland dysfunction on an average of at least 20 times per week. In certain embodiments, the patient experiences a symptom flare due to meibomian gland dysfunction on an average of at least 40 times per week. In certain embodiments, the patient experiences a symptom flare due to meibomian gland dysfunction on an average of from once per week to 15 times per week.

In certain embodiments, the patient has experienced at least 2, 3, 4, 5, 6, 7, 8, 9, 10, 11, 12, 13, 14, 15, 20, 25, or 30 symptom flares due to meibomian gland dysfunction during the month prior to first administration of the pharmaceutical composition. In certain embodiments, the patient has experienced at least five symptom flares due to meibomian gland dysfunction during the month prior to first administration of the pharmaceutical composition. In certain embodiments, the patient experiences a symptom flare due to meibomian gland dysfunction on an average of from 10 times per month to 30 times per month.

### Reduction in Meibomian Gland Dysfunction Symptoms

The method may be further characterized according to the reduction in meibomian gland dysfunction symptoms experienced by the patient. For example, in certain embodiments, the method produces a reduction in the number of symptom flares due to meibomian gland dysfunction. In certain embodiments, the method produces at least a 5%, 10%, 20%, 30%, 40%, 50%, 60%, 70%, 80%, 90%, or 100% reduction in the average number of symptom flares per month due to meibomian gland dysfunction compared to the average number of symptom flares due to meibomian gland dysfunction experienced by the patient in the month prior to first administering the pharmaceutical composition.

In certain embodiments, the method produces at least a 10% reduction in the average number of symptom flares per month due to meibomian gland dysfunction compared to the average number of symptom flares due to meibomian gland dysfunction experienced by the patient in the month prior to first administering the pharmaceutical composition. In certain embodiments, the method produces at least a 25% reduction in the average number of symptom flares per month due to meibomian gland dysfunction compared to the average number of symptom flares due to meibomian gland dysfunction experienced by the patient in the month prior to first administering the pharmaceutical composition. In certain embodiments, the method produces at least a 50% reduction in the average number of symptom flares per month due to meibomian gland dysfunction compared to the average number of symptom flares due to meibomian gland dysfunction experienced by the patient in the month prior to first administering the pharmaceutical composition.

In certain embodiments, as a result of the method, the average number of symptom flares experienced by the patient per month due to meibomian gland dysfunction is less than 50, 40, 30, 25, 20, 10, 5, or 1. In certain embodiments, as a result of the method, the average number of symptom flares experienced by the patient per month due to meibomian gland dysfunction is less than 10. In certain embodiments, as a result of the method, the average number of symptom flares experienced by the patient per month due to meibomian gland dysfunction is less than 4. In certain embodiments, as a result of the method, the average number of symptom flares experienced by the patient per month due to meibomian gland dysfunction is less than 2.

In certain embodiments, as a result of the method, the average number of symptom flares experienced by the patient per week due to meibomian gland dysfunction is less than 3. In certain embodiments, as a result of the method, the average number of symptom flares experienced by the patient per week due to meibomian gland dysfunction is less than 2. In certain embodiments, as a result of the method, the average number of symptom flares experienced by the patient per week due to meibomian gland dysfunction is less than 1.

In certain embodiments, the method produces a reduction in the number of symptom flares experienced by the patient per day due to meibomian gland dysfunction. In certain embodiments, as a result of the method, the average number of symptom flares experienced by the patient per day due to meibomian gland dysfunction is less than 2. In certain embodiments, as a result of the method, the average number of symptom flares experienced by the patient per day due to meibomian gland dysfunction is less than 1.

In certain embodiments, the method produces at least a 1%, 2%, 3%, 4%, 5%, 6%, 7%, 8%, 9%, 10%, 12%, 14%, 16%, 18%, 20%, 30%, 40%, 50%, 60%, 70%, 80%, 90%, or 100% reduction in the average number of symptom flares per month due to meibomian gland dysfunction compared to the average number of symptom flares per month prior to starting treatment using pharmaceutical composition. In certain embodiments, the method produces at least a 10% reduction in the average number of symptom flares per month due to meibomian gland dysfunction compared to the average number of symptom flares per month prior to starting treatment using pharmaceutical composition. In certain embodiments, the method produces at least a 50% reduction in the average number of symptom flares per month due to meibomian gland dysfunction compared to the average number of symptom flares per month prior to starting treatment using pharmaceutical composition.

In certain embodiments, the method produces at least a 1%, 2%, 3%, 4%, 5%, 6%, 7%, 8%, 9%, 10%, 12%, 14%, 16%, 18%, 20%, 30%, 40%, 50%, 60%, 70%, 80%, 90%, or 100% reduction in the average number of symptom flares per week due to meibomian gland dysfunction compared to the average number of symptom flares per week prior to starting treatment using pharmaceutical composition. In certain embodiments, the method produces at least a 10% reduction in the average number of symptom flares per week due to meibomian gland dysfunction compared to the average number of symptom flares per week prior to starting treatment using pharmaceutical composition. In certain embodiments, the method produces at least a 50% reduction in the average number of symptom flares per week due to meibomian gland dysfunction compared to the average number of symptom flares per week prior to starting treatment using pharmaceutical composition.

In certain embodiments, the method produces at least a 1%, 2%, 3%, 4%, 5%, 6%, 7%, 8%, 9%, 10%, 12%, 14%, 16%, 18%, 20%, 30%, 40%, 50%, 60%, 70%, 80%, 90%, or 100% reduction in the average number of symptom flares per day due to meibomian gland dysfunction compared to the average number of symptom flares per day prior to starting treatment using pharmaceutical composition. In certain embodiments, the method produces at least a 10% reduction in the average number of symptom flares per day due to meibomian gland dysfunction compared to the average number of symptom flares per day prior to starting treatment using pharmaceutical composition. In certain embodiments, the method produces at least a 50% reduction in the average number of symptom flares per day due to meibomian gland dysfunction compared to the average number of symptom flares per day prior to starting treatment using pharmaceutical composition.

Each of the above reductions in meibomian gland dysfunction symptoms experienced by the patient may be further characterized according to the duration of treatment received by the patient in order to achieve the reduction. For example, in certain embodiments, said reduction is achieved within twelve weeks after first administering the pharmaceutical composition. In certain embodiments, said reduction is achieved within 4, 5, 6, 7, 8, 9 or 10 weeks after first administering the pharmaceutical composition.

### M. Additional Exemplary Features of the Fourth, Eighth, and Tenth Therapeutic Methods

Additional exemplary features that may characterize the Fourth, Eighth, and Tenth Therapeutic Methods described herein are provided below and include, for example, features of the single therapeutic agent. A more thorough description of such features is provided below. The invention embraces all permutations and combinations of these features.

### Amount of Ethylene-Propylene-Styrene Copolymer

The methods may be further characterized according to the amount of ethylene-propylene-styrene copolymer in the single therapeutic agent. For example, in certain embodiments, ethylene-propylene-styrene copolymer is present in the single therapeutic agent in an amount of from about 0.01% (w/w) to about 10% (w/w) of the single therapeutic agent. in certain embodiments, ethylene-propylene-styrene copolymer is present in the single therapeutic agent in an amount of from about 0.1% (w/w) to about 10% (w/w) of the single therapeutic agent. In certain embodiments, ethylene-propylene-styrene copolymer is present in the single therapeutic agent in an amount of from about 0.05% (w/w) to about 8% (w/w) of the single therapeutic agent. In certain embodiments, ethylene-propylene-styrene copolymer is present in the single therapeutic agent in an amount of from about 1.75% (w/w) to about 7% (w/w) of the single therapeutic agent. In certain embodiments, ethylene-propylene-styrene copolymer is present in the single therapeutic agent in an amount of from about 0.1% (w/w) to about 6% (w/w) of the single therapeutic agent. In certain embodiments, ethylene-propylene-styrene copolymer is present in the single therapeutic agent in an amount of from about 3% (w/w) to about 7% (w/w) of the single therapeutic agent.

In certain embodiments, ethylene-propylene-styrene copolymer is present in the single therapeutic agent in an amount of from about 0.5% (w/w) to about 3% (w/w) of the single therapeutic agent. In certain embodiments, ethylene-propylene-styrene copolymer is present in the single therapeutic agent in an amount of from about 0.75% (w/w) to about 3% (w/w) of the single therapeutic agent. In certain embodiments, ethylene-propylene-styrene copolymer is present in the single therapeutic agent in an amount of from about 1% (w/w) to about 3% (w/w) of the single therapeutic agent. In certain embodiments, ethylene-propylene-styrene copolymer is present in the single therapeutic agent in an amount of from about 1% (w/w) to about 4% (w/w) of the single therapeutic agent.

### Amount of Butylene-Ethylene-Styrene Copolymer

The methods may be further characterized according to the amount of butylene-ethylene-styrene copolymer in the single therapeutic agent. For example, in certain embodiments, butylene-ethylene-styrene copolymer is present in the single therapeutic agent. In certain embodiments, butylene-ethylene-styrene copolymer is present in the single therapeutic agent in an amount of from about 0.001% (w/w) to about 3% (w/w) of the single therapeutic agent. In certain embodiments, butylene-ethylene-styrene copolymer is present in the single therapeutic agent in an amount of from about 0.01% (w/w) to about 3% (w/w) of the single therapeutic agent. In certain embodiments, butylene-ethylene-styrene copolymer is present in the single therapeutic agent in an amount of from about 0.01% (w/w) to about 1% (w/w) of the single therapeutic agent. In certain embodiments, butylene-ethylene-styrene copolymer is present in the single therapeutic agent in an amount of from about 0.03% (w/w) to about 0.75% (w/w) of the single therapeutic agent. In certain embodiments, butylene-ethylene-styrene copolymer is present in the single therapeutic agent in an amount of from about 0.01% (w/w) to about 0.2% (w/w) of the single therapeutic agent. In certain embodiments, butylene-ethylene-styrene copolymer is present in the single therapeutic agent in an amount of from about 0.07% (w/w) to about 1.75% (w/w) of the single therapeutic agent. In certain embodiments, butylene-ethylene-styrene copolymer is present in the single therapeutic agent in an amount of from about 0.1% (w/w) to about 0.5% (w/w) of the single therapeutic agent. In certain embodiments, butylene-ethylene-styrene copolymer is present in the single therapeutic agent in an amount of from about 0.01% (w/w) to about 0.5% (w/w) of the single therapeutic agent.

In certain embodiments, butylene-ethylene-styrene copolymer is present in the single therapeutic agent, but at an amount less than 0.75% (w/w) of the single therapeutic agent. In certain embodiments, butylene-ethylene-styrene copolymer is present in the single therapeutic agent, but at an amount less than 0.5% (w/w) of the single therapeutic agent. In certain embodiments, butylene-ethylene-styrene copolymer is present in the single therapeutic agent, but at an amount less than 0.4% (w/w) of the single therapeutic agent. In certain embodiments, butylene-ethylene-styrene copolymer is present in the single therapeutic agent, but at an amount less than 0.25% (w/w) of the single therapeutic agent.

### Amount of Mineral Oil

The methods may be further characterized according to the amount of mineral oil in the single therapeutic agent. For example, in certain embodiments, mineral oil is present in the single therapeutic agent. In certain embodiments, mineral oil is present in the single therapeutic agent in an amount of at least 90% (w/w) of the single therapeutic agent. In certain embodiments, mineral oil is present in the single therapeutic agent in an amount of at least 93% (w/w) of the single therapeutic agent. In certain embodiments, mineral oil is present in the single therapeutic agent in an amount of at least 95% (w/w) of the single therapeutic agent. In certain embodiments, mineral oil is present in the single therapeutic agent in an amount of at least 96% (w/w) of the single therapeutic agent. In certain embodiments, mineral oil is present in the single therapeutic agent in an amount of at least 97% (w/w) of the single therapeutic agent. In certain embodiments, mineral oil is present in the single therapeutic agent in an amount of at least 98% (w/w) of the single therapeutic agent. In certain embodiments, mineral oil is present in the single therapeutic agent in an amount of at least 99% (w/w) of the single therapeutic agent.

In certain embodiments, mineral oil is present in the single therapeutic agent in an amount of from 90% (w/w) to 99% (w/w) of the single therapeutic agent. In certain embodiments, mineral oil is present in the single therapeutic agent in an amount of from 93% (w/w) to 98% (w/w) of the single therapeutic agent. In certain embodiments, mineral oil is present in the single therapeutic agent in an amount of from 96% (w/w) to 99% (w/w) of the single therapeutic agent. In certain embodiments, mineral oil is present in the single therapeutic agent in an amount of from 96% (w/w) to 98% (w/w) of the single therapeutic agent. In certain embodiments, mineral oil is present in the single therapeutic agent in an amount of from 97% (w/w) to 98% (w/w) of the single therapeutic agent. In certain embodiments, mineral oil is present in the single therapeutic agent in an amount of from 98% (w/w) to 99% (w/w) of the single therapeutic agent.

### Exemplary More Specific Embodiments

The disclosure provides the following additional specific embodiments. Accordingly, in certain embodiments, the single therapeutic agent consists of:
(a) from about 1.75% (w/w) to about 7% (w/w) of an ethylene-propylene-styrene copolymer;
(b) from about 0.07% (w/w) to about 1.75% (w/w) of a butylene-ethylene-styrene copolymer; and
(c) mineral oil.

certain embodiments, the single therapeutic agent consists of:
(a) from about 2% (w/w) to about 7% (w/w) of an ethylene-propylene-styrene copolymer;
(b) from about 0.07% (w/w) to about 1% (w/w) of a butylene-ethylene-styrene copolymer; and
(c) mineral oil.

### Characteristics of Ethylene-Propylene-Styrene Copolymer

The methods may be further characterized according to characteristics of the ethylene-propylene-styrene copolymer in the single therapeutic agent. For example, in certain embodiments, the ethylene-propylene-styrene copolymer has a weight-average molecular weight in the range of from about 150,000 g/mol to about 250,000 g/mol. In certain embodiments, the ethylene-propylene-styrene copolymer has a weight-average molecular weight of about 200,000 g/mol.

In certain embodiments, the ethylene-propylene-styrene copolymer is a copolymer formed by polymerization of isoprene and styrene monomers that is terminated by hydrogenation. In certain embodiments, the ethylene-propylene-styrene copolymer is a copolymer formed by polymerization of isoprene and styrene followed by hydrogenation.

### Characteristics of Butylene-Ethylene-Styrene Copolymer

The methods may be further characterized according to characteristics of the butylene-ethylene-styrene copolymer in the single therapeutic agent. For example, in certain embodiments, the butylene-ethylene-styrene copolymer has a weight-average molecular weight in the range of from about 50,000 g/mol to about 150,000 g/mol. In certain embodiments, the butylene-ethylene-styrene copolymer has a weight-average molecular weight of about 100,000 g/mol.

In certain embodiments, the butylene-ethylene-styrene copolymer is a copolymer formed by polymerization of 1,3-butadiene and styrene monomers that is terminated by hydrogenation. In certain embodiments, the butylene-ethylene-styrene copolymer is a copolymer formed by polymerization of 1,3-butadiene and styrene followed by hydrogenation.

### Characteristics of Mineral Oil

The methods may be further characterized according to characteristics of the mineral oil in the single therapeutic agent. For example, in certain embodiments, the mineral oil has a weight-average molecular weight in the range of from about 100 g/mol to about 1,000 g/mol. In certain embodiments, the mineral oil has a weight-average molecular weight in the range of from about 200 g/mol to about 700 g/mol. In certain embodiments, the mineral oil has a weight-average molecular weight in the range of from about 230 g/mol to about 700 g/mol.

In certain embodiments, the mineral oil has a weight-average molecular weight in the range of from about 200 g/mol to about 500 g/mol. In certain embodiments, the mineral oil has a weight-average molecular weight in the range of from about 300 g/mol to about 600 g/mol. In certain embodiments, the mineral oil has a weight-average molecular weight in the range of from about 400 g/mol to about 700 g/mol.

In certain embodiments, the mineral oil has a weight-average molecular weight in the range of from about 200 g/mol to about 500 g/mol. In certain embodiments, the mineral oil has a weight-average molecular weight in the range of from about 300 g/mol to about 600 g/mol. In certain embodiments, the mineral oil has a weight-average molecular weight in the range of from about 400 g/mol to about 700 g/mol. In certain embodiments, the mineral oil has a weight-average molecular weight in the range of from about 400 g/mol to about 500 g/mol. In certain embodiments, the mineral oil has a weight-average molecular weight in the range of from about 440 g/mol to about 465 g/mol.

In certain embodiments, the mineral oil has a molecular weight in the range of from about 440 g/mol to about 465 g/mol. In certain embodiments, the mineral oil has a molecular weight of about 452 g/mol.

In certain embodiments, the mineral oil has a viscosity greater than 34.5 centistokes when measured at 40° C. In certain embodiments, the mineral oil has a viscosity in the range of from about 34.5 centistokes to about 150 centistokes when measured at 40° C. In certain embodiments, the mineral oil has a viscosity in the range of from about 34.5 centistokes to about 50 centistokes, from about 50 centistokes to about 75 centistokes, from about 75 centistokes to about 100 centistokes, from about 100 centistokes to about 125 centistokes, from about 125 centistokes to about 150 centistokes, or from about 34.5 centistokes to about 100 centistokes when measured at 40° C.

In certain embodiments, the mineral oil has a specific gravity of from about 0.845 to about 0.905. In certain embodiments, the mineral oil has a specific gravity of from about 0.8 to about 0.95. In certain embodiments, the mineral oil has a specific gravity of from about 0.8 to about 0.9. In certain embodiments, the mineral oil has a specific gravity of from about 0.84 to about 0.91. In certain embodiments, the mineral oil has a specific gravity of from about 0.845 to about 0.905, when measured at 20° C. In certain embodiments, the mineral oil has a specific gravity of from about 0.8 to about 0.95, when measured at 20° C. In certain embodiments, the mineral oil has a specific gravity of from about 0.8 to about 0.9, when measured at 20° C. In certain embodiments, the mineral oil has a specific gravity of from about 0.84 to about 0.91, when measured at 20° C. In certain embodiments, the mineral oil has a density of about 0.83 g/mL.

In certain embodiments, the mineral oil corresponds to the mineral oil identified by CAS registry number 8042-47-5.

In certain embodiments, the mineral oil has a viscosity less than 34.5 centistokes when measured at 40° C. In certain embodiments, the mineral oil has a viscosity less than 33.5 centistokes when measured at 40° C. In certain embodiments, the mineral oil has a viscosity in the range of from about 1 centistoke to about 34.4 centistokes when measured at 40° C. In certain embodiments, the mineral oil has a viscosity in the range of from about 1 centistoke to about 10 centistokes, from about 10 centistokes to about 20 centistokes, from about 20 centistokes to about 30 centistokes, or from about 25 centistokes to about 34.4 centistokes, when measured at 40° C.

In certain embodiments, the mineral oil has a specific gravity of from about 0.818 to about 0.88. In certain embodiments, the mineral oil has a specific gravity of from about 0.8 to about 0.9. In certain embodiments, the mineral oil has a specific gravity of from about 0.818 to about 0.88, when measured at 20° C. In certain embodiments, the mineral oil has a specific gravity of from about 0.8 to about 0.9, when measured at 20° C.

Formulation into a Pharmaceutical Composition

In certain embodiments, the single therapeutic agent is formulated into a pharmaceutical composition for administration to the patient. Such pharmaceutical compositions may be further characterized according to, for example, features described herein below.

In certain embodiments, the pharmaceutical composition is in the form of an ointment. In certain preferred embodiments, the pharmaceutical composition is in the form of a gel.

### N. Additional Exemplary Features of the First, Second, Third, Fourth, Fifth, Sixth, Seventh, Eighth, Nineth, Tenth, and Eleventh Therapeutic Methods

Additional exemplary features that may characterize the First, Second, Third, Fourth, Fifth, Sixth, Seventh, Eighth, Nineth, Tenth, and Eleventh Therapeutic Methods described herein are provided below and include, for example, features of the pharmaceutical composition, the dosing regimen used to administer the pharmaceutical composition to the patient, and results produced by the methods. A more thorough description of such features is provided below. The invention embraces all permutations and combinations of these features.

### 1. Pharmaceutical Composition

The method may be further characterized according to the composition of the pharmaceutical composition. For example, in certain embodiments, the pharmaceutical composition may be further characterized according to the amount of ethylene-propylene-styrene copolymer in the pharmaceutical composition. In certain embodiments, the pharmaceutical composition may be further characterized according to the amount of butylene-ethylene-styrene copolymer in the pharmaceutical composition. A more thorough description of such features is provided below. The invention embraces all permutations and combinations of these features.

### Amount of Ethylene-Propylene-Styrene Copolymer

The methods may be further characterized according to the amount of ethylene-propylene-styrene copolymer in the pharmaceutical composition. For example, in certain embodiments, ethylene-propylene-styrene copolymer is present in the pharmaceutical composition in an amount of from about 0.01% (w/w) to about 10% (w/w) of the pharmaceutical composition. In certain embodiments, ethylene-propylene-styrene copolymer is present in the pharmaceutical composition in an amount of from about 0.1% (w/w) to about 10% (w/w) of the pharmaceutical composition. In certain embodiments, ethylene-propylene-styrene copolymer is present in the pharmaceutical composition in an amount of from about 0.05% (w/w) to about 8% (w/w) of the pharmaceutical composition. In certain embodiments, ethylene-propylene-styrene copolymer is present in the pharmaceutical composition in an amount of from about 0.1% (w/w) to about 6% (w/w) of the pharmaceutical composition. In certain embodiments, ethylene-propylene-styrene copolymer is present in the pharmaceutical composition in an amount of from about 1.75% (w/w) to about 7% (w/w) of the pharmaceutical composition. In certain embodiments, ethylene-propylene-styrene copolymer is present in the pharmaceutical composition in an amount of from about 3% (w/w) to about 10% (w/w) of the pharmaceutical composition. In certain embodiments, ethylene-propylene-styrene copolymer is present in the pharmaceutical composition in an amount of from about 3% (w/w) to about 7% (w/w) of the pharmaceutical composition.

In certain embodiments, ethylene-propylene-styrene copolymer is present in the pharmaceutical composition in an amount of from about 0.5% (w/w) to about 3% (w/w) of the pharmaceutical composition. In certain embodiments, ethylene-propylene-styrene copolymer is present in the pharmaceutical composition in an amount of from about 0.75% (w/w) to about 3% (w/w) of the pharmaceutical composition. In certain embodiments, ethylene-propylene-styrene copolymer is present in the pharmaceutical composition in an amount of from about 1% (w/w) to about 3% (w/w) of the pharmaceutical composition. In certain embodiments, ethylene-propylene-styrene copolymer is present in the pharmaceutical composition in an amount of from about 1% (w/w) to about 4% (w/w) of the pharmaceutical composition.

### Amount of Butylene-Ethylene-Styrene Copolymer

The methods may be further characterized according to the amount of butylene-ethylene-styrene copolymer in the pharmaceutical composition. For example, in certain embodiments, butylene-ethylene-styrene copolymer is present in the pharmaceutical composition. In certain embodiments, butylene-ethylene-styrene copolymer is present in the pharmaceutical composition in an amount of from about 0.001% (w/w) to about 3% (w/w) of the pharmaceutical composition. In certain embodiments, butylene-ethylene-styrene copolymer is present in the pharmaceutical composition in an amount of from about 0.01% (w/w) to about 3% (w/w) of the pharmaceutical composition. In certain embodiments, butylene-ethylene-styrene copolymer is present in the pharmaceutical composition in an amount of from about 0.01% (w/w) to about 1% (w/w) of the pharmaceutical composition. In certain embodiments, butylene-ethylene-styrene copolymer is present in the pharmaceutical composition in an amount of from about 0.03% (w/w) to about 0.75% (w/w) of the pharmaceutical composition. In certain embodiments, butylene-ethylene-styrene copolymer is present in the pharmaceutical composition in an amount of from about 0.01% (w/w) to about 0.2% (w/w) of the pharmaceutical composition. In certain embodiments, butylene-ethylene-styrene copolymer is present in the pharmaceutical composition in an amount of from about 0.07% (w/w) to about 1.75% (w/w) of the pharmaceutical composition. In certain embodiments, butylene-ethylene-styrene copolymer is present in the pharmaceutical composition in an amount of from about 0.1% (w/w) to about 0.5% (w/w) of the pharmaceutical composition. In certain embodiments, butylene-ethylene-styrene copolymer is present in the pharmaceutical composition in an amount of from about 0.01% (w/w) to about 0.5% (w/w) of the pharmaceutical composition.

In certain embodiments, butylene-ethylene-styrene copolymer is present in the pharmaceutical composition, but at an amount less than 0.75% (w/w) of the pharmaceutical composition. In certain embodiments, butylene-ethylene-styrene copolymer is present in the pharmaceutical composition, but at an amount less than 0.5% (w/w) of the pharmaceutical composition. In certain embodiments, butylene-ethylene-styrene copolymer is present in the pharmaceutical composition, but at an amount less than 0.4% (w/w) of the pharmaceutical composition. In certain embodiments, butylene-ethylene-styrene copolymer is present in the pharmaceutical composition, but at an amount less than 0.25% (w/w) of the pharmaceutical composition.

### Amount of Mineral Oil

The methods may be further characterized according to the amount of mineral oil in the pharmaceutical composition. For example, in certain embodiments, mineral oil is present in the pharmaceutical composition. In certain embodiments, mineral oil is present in the pharmaceutical composition in an amount of at least 90% (w/w) of the pharmaceutical composition. In certain embodiments, mineral oil is present in the pharmaceutical composition in an amount of at least 93% (w/w) of the pharmaceutical composition. In certain embodiments, mineral oil is present in the pharmaceutical composition in an amount of at least 95% (w/w) of the pharmaceutical composition. In certain embodiments, mineral oil is present in the pharmaceutical composition in an amount of at least 96% (w/w) of the pharmaceutical composition. In certain embodiments, mineral oil is present in the pharmaceutical composition in an amount of at least 97% (w/w) of the pharmaceutical composition. In certain embodiments, mineral oil is present in the pharmaceutical composition in an amount of at least 98% (w/w) of the pharmaceutical composition. In certain embodiments, mineral oil is present in the pharmaceutical composition in an amount of at least 99% (w/w) of the pharmaceutical composition.

In certain embodiments, mineral oil is present in the pharmaceutical composition in an amount of from 90% (w/w) to 99% (w/w) of the pharmaceutical composition. In certain embodiments, mineral oil is present in the pharmaceutical composition in an amount of from 93% (w/w) to 98% (w/w) of the pharmaceutical composition. In certain embodiments, mineral oil is present in the pharmaceutical composition in an amount of from 93% (w/w) to 97% (w/w) of the pharmaceutical composition. In certain embodiments, mineral oil is present in the pharmaceutical composition in an amount of from 92% (w/w) to 97% (w/w) of the pharmaceutical composition. In certain embodiments, mineral oil is present in the pharmaceutical composition in an amount of from 96% (w/w) to 99% (w/w) of the pharmaceutical composition. In certain embodiments, mineral oil is present in the pharmaceutical composition in an amount of from 96% (w/w) to 98% (w/w) of the pharmaceutical composition. In certain embodiments, mineral oil is present in the pharmaceutical composition in an amount of from 97% (w/w) to 98% (w/w) of the pharmaceutical composition. In certain embodiments, mineral oil is present in the pharmaceutical composition in an amount of from 98% (w/w) to 99% (w/w) of the pharmaceutical composition.

### Amount of Antioxidant

The methods may be further characterized according to the amount of antioxidant in the pharmaceutical composition. For example, in certain embodiments, an antioxidant is present in the pharmaceutical composition. In certain embodiments, an antioxidant is present in the pharmaceutical composition in an amount of from about 0.001% (w/w) to about 0.5% (w/w) of the pharmaceutical composition. In certain embodiments, an antioxidant is present in the pharmaceutical composition in an amount of from about 0.01% (w/w) to about 0.2% (w/w) of the pharmaceutical composition.

In certain embodiments, an antioxidant is present in the pharmaceutical composition, but at an amount less than 0.5% (w/w) of the pharmaceutical composition. In certain embodiments, an antioxidant is present in the pharmaceutical composition, but at an amount less than 0.2% (w/w) of the pharmaceutical composition.

### Exemplary More Specific Embodiments

The disclosure provides the following additional specific embodiments. Accordingly, in certain embodiments, the pharmaceutical composition consists of:
(a) an ethylene-propylene-styrene copolymer;
(b) a butylene-ethylene-styrene copolymer;
(c) mineral oil;
(d) an antioxidant; and
(e) optionally one or more pharmaceutically acceptable carriers.

In certain embodiments, the pharmaceutical composition consists of:
(a) from about 1.75% (w/w) to about 7% (w/w) of an ethylene-propylene-styrene copolymer;
(b) from about 0.07% (w/w) to about 1.75% (w/w) of a butylene-ethylene-styrene copolymer;
(c) mineral oil;
(d) an antioxidant; and
(e) optionally one or more pharmaceutically acceptable carriers.

In certain embodiments, the pharmaceutical composition consists of:
(a) from about 1.75% (w/w) to about 7% (w/w) of an ethylene-propylene-styrene copolymer;
(b) from about 0.07% (w/w) to about 1.75% (w/w) of a butylene-ethylene-styrene copolymer;
(c) mineral oil;
(d) from about 0.001% (w/w) to about 0.2% (w/w) of an antioxidant; and
(e) optionally one or more pharmaceutically acceptable carriers.

In certain embodiments, the pharmaceutical composition consists of:
(a) from about 1.75% (w/w) to about 7% (w/w) of an ethylene-propylene-styrene copolymer;
(b) from about 0.07% (w/w) to about 1.75% (w/w) of a butylene-ethylene-styrene copolymer;
(c) mineral oil; and
(d) an antioxidant.

In certain embodiments, the pharmaceutical composition consists of:
(a) from about 1.75% (w/w) to about 7% (w/w) of an ethylene-propylene-styrene copolymer;
(b) from about 0.07% (w/w) to about 1.75% (w/w) of a butylene-ethylene-styrene copolymer;
(c) mineral oil; and
(d) from about 0.001% (w/w) to about 0.2% (w/w) of an antioxidant.

### Characteristics of Ethylene-Propylene-Styrene Copolymer

The methods may be further characterized according to characteristics of the ethylene-propylene-styrene copolymer in the pharmaceutical composition. For example, in certain embodiments, the ethylene-propylene-styrene copolymer has a weight-average molecular weight in the range of from about 150,000 g/mol to about 250,000 g/mol. In certain embodiments, the ethylene-propylene-styrene copolymer has a weight-average molecular weight of about 200,000 g/mol.

In certain embodiments, the ethylene-propylene-styrene copolymer is a copolymer formed by polymerization of isoprene and styrene monomers that is terminated by hydrogenation. In certain embodiments, the ethylene-propylene-styrene copolymer is a copolymer formed by polymerization of isoprene and styrene followed by hydrogenation.

### Characteristics of Butylene-Ethylene-Styrene Copolymer

The methods may be further characterized according to characteristics of the butylene-ethylene-styrene copolymer in the pharmaceutical composition. For example, in certain embodiments, the butylene-ethylene-styrene copolymer has a weight-average molecular weight in the range of from about 50,000 g/mol to about 150,000 g/mol. In certain embodiments, the butylene-ethylene-styrene copolymer has a weight-average molecular weight of about 100,000 g/mol.

In certain embodiments, the butylene-ethylene-styrene copolymer is a copolymer formed by polymerization of 1,3-butadiene and styrene monomers that is terminated by hydrogenation. In certain embodiments, the butylene-ethylene-styrene copolymer is a copolymer formed by polymerization of 1,3-butadiene and styrene followed by hydrogenation.

### Identity of Antioxidant

The methods may be further characterized according to the identity of the antioxidant in the pharmaceutical composition. For example, in certain embodiments, the antioxidant is a phenol. In certain embodiments, the antioxidant is a phenol substituted with at least one alkyl group. In certain embodiments, the antioxidant is butylated-hydroxytoluene.

### Characteristics of Mineral Oil

The methods may be further characterized according to characteristics of the mineral oil in the pharmaceutical composition. For example, in certain embodiments, the mineral oil has a weight-average molecular weight in the range of from about 100 g/mol to about 1,000 g/mol. In certain embodiments, the mineral oil has a weight-average molecular weight in the range of from about 200 g/mol to about 700 g/mol. In certain embodiments, the mineral oil has a weight-average molecular weight in the range of from about 230 g/mol to about 700 g/mol.

In certain embodiments, the mineral oil has a weight-average molecular weight in the range of from about 200 g/mol to about 500 g/mol. In certain embodiments, the mineral oil has a weight-average molecular weight in the range of from about 300 g/mol to about 600 g/mol. In certain embodiments, the mineral oil has a weight-average molecular weight in the range of from about 400 g/mol to about 700 g/mol. In certain embodiments, the mineral oil has a weight-average molecular weight in the range of from about 400 g/mol to about 500 g/mol. In certain embodiments, the mineral oil has a weight-average molecular weight in the range of from about 440 g/mol to about 465 g/mol.

In certain embodiments, the mineral oil has a molecular weight in the range of from about 440 g/mol to about 465 g/mol. In certain embodiments, the mineral oil has a molecular weight of about 452 g/mol.

In certain embodiments, the mineral oil has a viscosity greater than 34.5 centistokes when measured at 40° C. In certain embodiments, the mineral oil has a viscosity in the range of from about 34.5 centistokes to about 150 centistokes when measured at 40° C. In certain embodiments, the mineral oil has a viscosity in the range of from about 34.5 centistokes to about 50 centistokes, from about 50 centistokes to about 75 centistokes, from about 75 centistokes to about 100 centistokes, from about 100 centistokes to about 125 centistokes, from about 125 centistokes to about 150 centistokes, or from about 34.5 centistokes to about 100 centistokes when measured at 40° C.

In certain embodiments, the mineral oil has a specific gravity of from about 0.845 to about 0.905. In certain embodiments, the mineral oil has a specific gravity of from about 0.8 to about 0.95. In certain embodiments, the mineral oil has a specific gravity of from about 0.8 to about 0.9. In certain embodiments, the mineral oil has a specific gravity of from about 0.84 to about 0.91. In certain embodiments, the mineral oil has a specific gravity of from about 0.845 to about 0.905, when measured at 20° C. In certain embodiments, the mineral oil has a specific gravity of from about 0.8 to about 0.95, when measured at 20° C. In certain embodiments, the mineral oil has a specific gravity of from about 0.8 to about 0.9, when measured at 20° C. In certain embodiments, the mineral oil has a specific gravity of from about 0.84 to about 0.91, when measured at 20° C. In certain embodiments, the mineral oil has a density of about 0.83 g/mL.

In certain embodiments, the mineral oil corresponds to the mineral oil identified by CAS registry number 8042-47-5.

In certain embodiments, the mineral oil has a viscosity less than 34.5 centistokes when measured at 40° C. In certain embodiments, the mineral oil has a viscosity less than 33.5 centistokes when measured at 40° C. In certain embodiments, the mineral oil has a viscosity in the range of from about 1 centistoke to about 34.4 centistokes when measured at 40° C. In certain embodiments, the mineral oil has a viscosity in the range of from about 1 centistoke to about 10 centistokes, from about 10 centistokes to about 20 centistokes, from about 20 centistokes to about 30 centistokes, or from about 25 centistokes to about 34.4 centistokes, when measured at 40° C.

In certain embodiments, the mineral oil has a specific gravity of from about 0.818 to about 0.88. In certain embodiments, the mineral oil has a specific gravity of from about 0.8 to about 0.9. In certain embodiments, the mineral oil has a specific gravity of from about 0.818 to about 0.88, when measured at 20° C. In certain embodiments, the mineral oil has a specific gravity of from about 0.8 to about 0.9, when measured at 20° C.

### Physical Form of the Pharmaceutical Composition

The methods may be further characterized according to the physical form of the pharmaceutical composition. For example, in certain preferred embodiments, the pharmaceutical composition is in the form of a gel. In certain embodiments, the pharmaceutical composition is in the form of an ointment.

### Alternative Pharmaceutical Compositions

The following alternative pharmaceutical compositions may be used in the therapeutic methods described herein. The alternative pharmaceutical compositions are prepared by mixing a polymeric hydrocarbon gelling agent and mineral oil. Exemplary polymeric hydrocarbon gelling agents are described in more detail below.

In certain embodiments, the polymeric hydrocarbon gelling agent comprises an ethylene-propylene-styrene copolymer. In certain embodiments, the polymeric hydrocarbon gelling agent comprises an ethylene-propylene-styrene copolymer having a weight-average molecular weight in the range of from about 150,000 g/mol to about 250,000 g/mol. In certain embodiments, the polymeric hydrocarbon gelling agent comprises an ethylene-propylene-styrene copolymer having a weight-average molecular weight of about 200,000 g/mol. In certain embodiments, the polymeric hydrocarbon gelling agent comprises from about 1% (w/w) to about 15% (w/w) of ethylene-propylene-styrene copolymer. In certain embodiments, the polymeric hydrocarbon gelling agent comprises from about 2.5% (w/w) to about 10% (w/w) of ethylene-propylene-styrene copolymer.

In certain embodiments, the ethylene-propylene-styrene copolymer is a copolymer formed by polymerization of isoprene and styrene monomers that is terminated by hydrogenation. In certain embodiments, the ethylene-propylene-styrene copolymer is a copolymer formed by polymerization of isoprene and styrene followed by hydrogenation.

In certain embodiments, the polymeric hydrocarbon gelling agent further comprises a butylene-ethylene-styrene copolymer. In certain embodiments, the polymeric hydrocarbon gelling agent further comprises a butylene-ethylene-styrene copolymer having a weight-average molecular weight in the range of from about 50,000 g/mol to about 150,000 g/mol. In certain embodiments, the polymeric hydrocarbon gelling agent further comprises a butylene-ethylene-styrene copolymer having a weight-average molecular weight of about 100,000 g/mol. In certain embodiments, the polymeric hydrocarbon gelling agent comprises from about 0.01% (w/w) to about 2.5% (w/w) of butylene-ethylene-styrene copolymer. In certain embodiments, the polymeric hydrocarbon gelling agent comprises from about 0.1% (w/w) to about 2.5% (w/w) of butylene-ethylene-styrene copolymer.

In certain embodiments, the butylene-ethylene-styrene copolymer is a copolymer formed by polymerization of 1,3-butadiene and styrene monomers that is terminated by hydrogenation. In certain embodiments, the butylene-ethylene-styrene copolymer is a copolymer formed by polymerization of 1,3-butadiene and styrene followed by hydrogenation.

In certain embodiments, the polymeric hydrocarbon gelling agent further comprises butylated-hydroxytoluene. In certain embodiments, the polymeric hydrocarbon gelling agent further comprises from about 0.01% (w/w) to about 0.5% (w/w) of butylated-hydroxytoluene. In certain embodiments, the polymeric hydrocarbon gelling agent further comprises butylated-hydroxytoluene in an amount less than 0.5% (w/w).

In certain embodiments, the polymeric hydrocarbon gelling agent further comprises mineral oil. In certain embodiments, the polymeric hydrocarbon gelling agent further comprises at least about 80% (w/w) mineral oil. In certain embodiments, the polymeric hydrocarbon gelling agent further comprises at least about 90% (w/w) mineral oil. In certain embodiments, the mineral oil component of the polymeric hydrocarbon gelling agent has a weight-average molecular weight in the range of from about 100 g/mol to about 1,000 g/mol. In certain embodiments, the mineral oil component of the polymeric hydrocarbon gelling agent has a weight-average molecular weight in the range of from about 200 g/mol to about 700 g/mol. In certain embodiments, the mineral oil is white mineral oil.

The alternative pharmaceutical composition may be further characterized according to the amount of polymeric hydrocarbon gelling agent in the pharmaceutical composition. For example, in certain embodiments, the alternative pharmaceutical composition comprises from about 60% (w/w) to about 80% (w/w) of the polymeric hydrocarbon gelling agent. In certain embodiments, the alternative pharmaceutical composition comprises from about 65% (w/w) to about 75% (w/w) of the polymeric hydrocarbon gelling agent. In certain embodiments, the alternative pharmaceutical composition comprises from about 67% (w/w) to about 71% (w/w) of the polymeric hydrocarbon gelling agent. In certain embodiments, the alternative pharmaceutical composition comprises about 70% (w/w) of the polymeric hydrocarbon gelling agent. In certain embodiments, the alternative pharmaceutical composition comprises about 69% (w/w) of the polymeric hydrocarbon gelling agent.

In certain embodiments, the polymeric hydrocarbon gelling agent comprises:
(a) at least 80% (w/w) mineral oil;
(b) from about 2.5% (w/w) to about 10% (w/w) of ethylene-propylene-styrene copolymer; and
(c) comprises from about 0.1% (w/w) to about 2.5% (w/w) of butylene-ethylene-styrene copolymer.

The disclosure provides the following additional specific embodiments. Accordingly, in certain embodiments, the polymeric hydrocarbon gelling agent comprises:
(a) at least 80% (w/w) mineral oil having a weight-average molecular weight in the range of from about 100 g/mol to about 1,000 g/mol;
(b) from about 2.5% (w/w) to about 10% (w/w) of ethylene-propylene-styrene copolymer having a weight-average molecular weight of about 200,000 g/mol; and
(c) comprises from about 0.1% (w/w) to about 2.5% (w/w) of butylene-ethylene-styrene copolymer having a weight-average molecular weight of about 100,000 g/mol.

In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity in the range of from about 13,000 to about 28,000 cps at 25° C., as sold by Calumet Specialty Products Partners, L.P. under the tradename VERSAGEL® M200. In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity of about 20,0000 cps at 25° C., as sold by Calumet Specialty Products Partners, L.P. under the tradename VERSAGEL® M200.

In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity in the range of from about 47,000 to about 57,000 cps at 25° C., as sold by Calumet Specialty Products Partners, L.P. under the tradename VERSAGEL® M500. In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity of about 50,0000 cps at 25° C., as sold by Calumet Specialty Products Partners, L.P. under the tradename VERSAGEL® M500.

In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity in the range of from about 67,000 to about 83,000 cps at 25° C., as sold by Calumet Specialty Products Partners, L.P. under the tradename VERSAGEL® M750. In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity of about 75,000 cps at 25° C., as sold by Calumet Specialty Products Partners, L.P. under the tradename VERSAGEL® M750.

In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity of about 160,0000 cps at 25° C., as sold by Calumet Specialty Products Partners, L.P. under the tradename VERSAGEL® M1600.

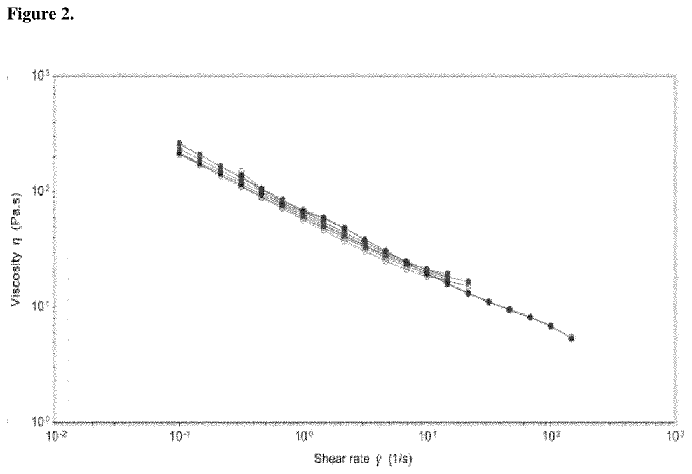

In certain embodiments, the polymeric hydrocarbon gelling agent may be further characterized according to its physical properties, such as (i) performance in a shear stress vs. shear rate analysis, (ii) yield stress analysis, and/or (iii) normal stress analysis. Results of an exemplary shear stress vs. shear rate analysis are displayed in FIG. 2 for the polymeric hydrocarbon gelling agent sold commercially under the tradename VERSAGEL® M-750, which is a mixture of ethylene-propylene-styrene copolymer, butylene-ethylene-styrene copolymer, butylated-hydroxytoluene, and mineral oil. The ethylene-propylene-styrene copolymer (having a weight-average molecular weight of about 200,000 g/mol) was present in an amount within the range of 2.5% to 10% (w/w), the butylene-ethylene-styrene copolymer (having a weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol). In certain embodiments, the polymeric hydrocarbon gelling agent displays substantially the same performance in shear stress vs. shear rate analysis as illustrated in FIG. 12.

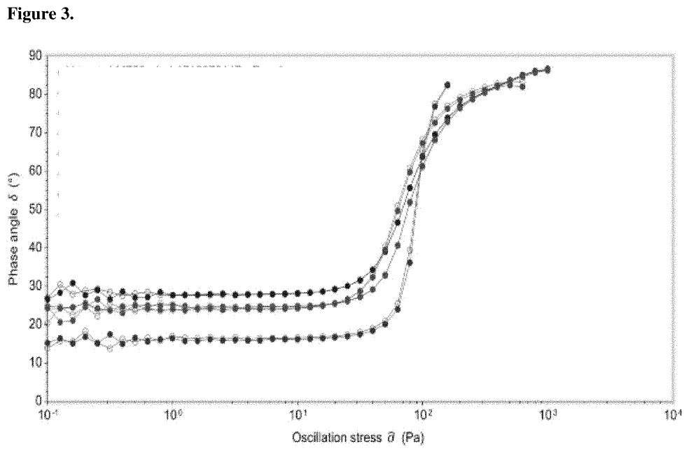

Results of an exemplary yield stress analysis are displayed in FIG. 3 for the polymeric hydrocarbon gelling agent sold commercially under the tradename VERSAGEL® M-750, which is as described above. In certain embodiments, the polymeric hydrocarbon gelling agent displays substantially the same performance in yield stress analysis as illustrated in FIG. 3.

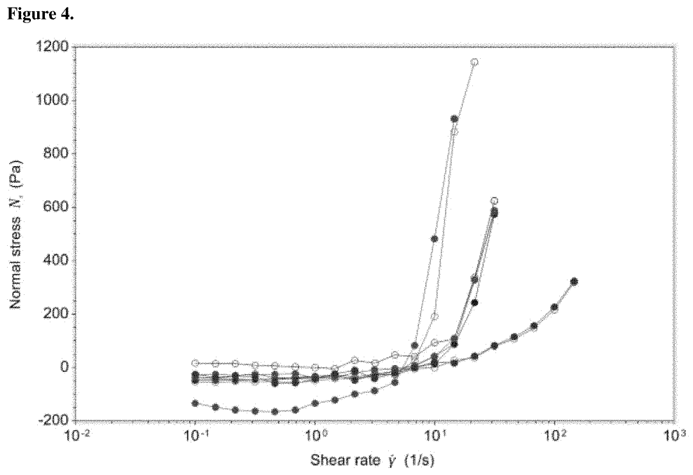

Results of an exemplary normal stress analysis are displayed in FIG. 4 for the polymeric hydrocarbon gelling agent sold commercially under the tradename VERSAGEL® M-750, which is as described above. In certain embodiments, the polymeric hydrocarbon gelling agent displays substantially the same performance in normal stress analysis as illustrated in FIG. 4.

In certain embodiments, an alternative pharmaceutical composition is prepared by mixing from about 2 parts to about 3 parts polymeric hydrocarbon gelling agent with about 1 part mineral oil. In certain embodiments, the alternative pharmaceutical compositions are prepared by mixing from about 2.2 parts to about 2.4 parts polymeric hydrocarbon gelling agent with about 1 part mineral oil. In certain embodiments, the alternative pharmaceutical compositions are prepared by mixing about 2.3 parts polymeric hydrocarbon gelling agent with about 1 part mineral oil. In certain embodiments, the alternative pharmaceutical composition comprises about 70% (w/w) polymeric hydrocarbon gelling agent and about 30% (w/w) mineral oil.

### Sterility Level of the Pharmaceutical Composition

The method may be further characterized according to the sterility of the pharmaceutical composition used. For example, in certain embodiments, the pharmaceutical composition has undergone sterilization, such as by exposing the pharmaceutical composition to gamma or e-beam sterilization. The level of sterility of the pharmaceutical composition may be characterized, e.g., where the pharmaceutical composition has a sterility assurance level that is more sterile than 10⁻¹, 10⁻², 10⁻³, 10⁻⁴, 10⁻⁵, 10⁻⁶, 10⁻⁷, 10⁻⁸, or 10⁻⁹. In certain embodiments, the pharmaceutical composition has a sterility assurance level of from about 10⁻¹ to 10⁻³, about 10⁻³ to about 10⁻⁴, about 10⁻⁴ to about 10⁻⁵, about 10⁻⁵ to about 10⁻⁶, or about 10⁻⁶ to about 10⁻⁷, or a sterility assurance level that is more sterile than 10⁻⁷. In certain embodiments, the pharmaceutical composition has a sterility assurance level of about 10⁻⁶.

### Non-Newtonian Physical Properties

The pharmaceutical composition desirably displays non-Newtonian physical properties. That is, the pharmaceutical composition is a non-Newtonian fluid. Such non-Newtonian physical properties provide superior residence time on the eyelid margin when the pharmaceutical composition is applied to the eyelid margin. Such non-Newtonian physical properties also minimize exposure of the cornea surface to the pharmaceutical composition when the pharmaceutical composition is applied to the eyelid margin.

A desired non-Newtonian physical property is where the pharmaceutical composition undergoes a reduction in viscosity due to mechanical forces imposed on the pharmaceutical composition due to the patient blinking their eye.

Viscosity of the pharmaceutical composition can be measured a different shear rates. For example, in certain embodiments, at a shear rate of 6 (1/s), the pharmaceutical composition has a viscosity in the range of from about 1,000 cP to about 45,000 cP, from about 3,000 cP to about 30,000 cP, from about 3,000 cP to about 25,000 cP, from about 3,000 cP to about 20,000 cP, from about 3,000 cP to about 15,000 cP, from about 5,000 cP to about 30,000 cP, from about 5,000 cP to about 25,000 cP, from about 5,000 cP to about 20,000 cP, from about 5,000 cP to about 15,000 cP, from about 6,000 cP to about 20,000 cP. In certain embodiments, at a shear rate of 6 (1/s), the pharmaceutical composition has a viscosity in the range of from about 7,000 cP to about 15,000 cP.

The pharmaceutical composition can also be characterized according to Oscillatory Stress Sweep, Oscillatory Frequency Sweep, Yield stress, Complex Modulus, and Loss Modulus. Additionally, the pharmaceutical composition can also be characterized according to normal stress test, which monitors the normal stress exhibited at a range of shear rates.

### 2. Dosage Considerations

The method may be further characterized according, for example, to the dosage, location to which the dosage is administered on the patient, and timing for the administration of the pharmaceutical composition to the patient.

### Dosing Amounts

The method may be further characterized according to the dosing amount of the pharmaceutical composition. For example, in certain embodiments, an amount of from about 35 μL to about 65 μL of the pharmaceutical composition is topically administered to the eyelid margin of the patient. In certain embodiments, an amount of from about 40 μL to about 50 μL of the pharmaceutical composition is topically administered to the eyelid margin of the patient. In certain embodiments, an amount of from about 45 μL to about 55 μL of the pharmaceutical composition is topically administered to the eyelid margin of the patient. In certain embodiments, an amount of about 50 μL of the pharmaceutical composition is topically administered to the eyelid margin of the patient.

In certain embodiments, the pharmaceutical composition is topically administered to the eyelid margin of the patient using either a fingertip, application directly from a container containing the pharmaceutical composition, or a device for application of the pharmaceutical composition.

### Location for Administration

The method may be further characterized according to the location for administration of the pharmaceutical composition. For example, in certain embodiments, the pharmaceutical composition is topically administered to the eyelid margin of the patient to form a strip having a width less than or equal to one-quarter inches. In certain embodiments, the pharmaceutical composition is topically administered to the eyelid margin of the patient to form a strip having a width of about one-quarter inches. In certain embodiments, the pharmaceutical composition is topically administered across the full margin of the eyelid.

When administering the pharmaceutical composition to the eyelid margin, one embodiment is for the patient to pull down the lower eyelid and look up, then use their finger to apply the pharmaceutical composition (e.g., a pea sized amount of pharmaceutical composition) to the inside of the lower eyelid, between the lower eyelid and the eye. The patient may optionally apply the pharmaceutical composition as a thin ribbon on the lower eyelid close to their nose (inner canthus) and direct outward without touching the eyelash or the eye. The ribbon of the pharmaceutical composition is desirably deposited on the inside lining of the lower eyelid.

Application of the pharmaceutical composition to the eyelid margin desirably brings the pharmaceutical composition into contact with one or more of the tarsal conjunctiva, conjunctival fornix, bulbar conjunctiva, or conjunctival sac. This disclosure provides methods where, in lieu of applying the pharmaceutical composition as a strip across the eyelid margin, the pharmaceutical composition is administered directly to one or more of the tarsal conjunctiva, conjunctival fornix, bulbar conjunctiva, or conjunctival sac.

In certain embodiments, the pharmaceutical composition is administered to the eyelid margin of the patient using an applicator. In certain embodiments, the pharmaceutical composition is administered to the eyelid margin of the patient using a fingertip. In certain embodiments, the pharmaceutical composition is administered to the eyelid margin of the patient using a container or vessel containing the pharmaceutical composition.

### Frequency of Administration

The method may be further characterized according to the frequency of administration of the pharmaceutical composition. For example, in certain embodiments, the pharmaceutical composition is administered twice per day. In certain embodiments, the pharmaceutical composition is administered twice per day, wherein a first dose of pharmaceutical composition is administered in the morning and a second dose of pharmaceutical composition is administered in the evening. In certain embodiments, the pharmaceutical composition is administered twice per day, wherein there is from about 8 hours to about 12 hours between administering a first dose of pharmaceutical composition and administering a second dose of pharmaceutical composition. In certain embodiments, the pharmaceutical composition is administered twice per day, wherein there is at least about 8 hours between administering a first dose of pharmaceutical composition and administering a second dose of pharmaceutical composition. In certain embodiments, the pharmaceutical composition is administered up to twice per day as needed.

In certain embodiments, the pharmaceutical composition is administered once per day. In certain embodiments, the pharmaceutical composition is administered once per day, in the evening. In certain embodiments, the pharmaceutical composition is administered once per day, at or near bedtime of the patient.

In certain embodiments, for a duration of at least thirty days the patient receives a dose of the pharmaceutical composition each day. In certain embodiments, for a duration of at least two months the patient receives a dose of the pharmaceutical composition each day. In certain embodiments, for a duration of at least three months the patient receives a dose of the pharmaceutical composition each day. In certain embodiments, for a duration of at least six months the patient receives a dose of the pharmaceutical composition each day. In certain embodiments, for a duration of at least twelve months the patient receives a dose of the pharmaceutical composition each day.

3. Patient Populations that May Derive Particular Benefits from the Therapeutic Methods

The methods may be further characterized according to the patient to be treated. For example, in certain embodiments, the patient is a human. In certain embodiments, the patient is an adult human.

### Tear Osmolarity

The methods may be further characterized according to the tear osmolarity of the patient to be treated. For example, in certain embodiments, the patient has a tear osmolarity value greater than about 315 mOsmol/L. In certain embodiments, the patient has a tear osmolarity value greater than about 310, 312, 315, 320, 325, or 330 mOsmol/L. In certain embodiments, the patient has a tear osmolarity value in the range of from about 310 mOsmol/L to 330 mOsmol/L. In certain embodiments, the patient has a tear osmolarity value in the range of from about 310 mOsmol/L to 315 mOsmol/L. In certain embodiments, the patient has a tear osmolarity value in the range of from about 315 mOsmol/L to 330 mOsmol/L.

### Tear Film Matrix Metalloproteinase-9 (MMP-9) Concentration

The methods may be further characterized according to the tear film Matrix metalloproteinase-9 (MMP-9) concentration of the patient to be treated. For example, in certain embodiments, the patient's tear film has a concentration of MMP-9 less than 40 ng/mL. In certain embodiments, the patient's tear film has a concentration of MMP-9 less than about 35, 30, 25, 20, 15, 10, or 5 ng/mL. In certain embodiments, the patient's tear film has a concentration of MMP-9 in the range of from 0 ng/mL to less than 40 ng/mL. In certain embodiments, the patient's tear film has a concentration of MMP-9 in the range of from about 15 ng/mL to less than 40 ng/mL.

### Tear Film Breakup Time

The methods may be further characterized according to the tear film breakup time of the patient to be treated. For example, in certain embodiments, the patient's tear film breakup time has a breakup time of less than 10 seconds. For example, in certain embodiments, the tear film break up time is less than 8 seconds, 6 seconds, 4 seconds, or 2 seconds. In certain embodiments, the tear film break up time is in the range of 1 second to 10 seconds. In certain embodiments, the tear film break up time is in the range of 1 second to 5 seconds. In certain embodiments, the tear film break up time is in the range of 1 second to 4 seconds. In certain embodiments, the tear film break up time is in the range of 1 second to 2 seconds.

### 4. Therapeutic Improvements & Other Characteristics

The methods may be further characterized according to therapeutic benefits. Exemplary therapeutic benefits that may be measured are described herein below.

### Reduction in Vascular Engorgement

The methods may be further characterized according to the reduction in vascular engorgement experienced by the patient. For example, in certain embodiments, the method produces a reduction in vascular engorgement score of at least 1. In certain embodiments, the method produces a reduction in vascular engorgement score of at least 2. For example, in certain embodiments, the method produces a reduction in vascular engorgement score of at least 0.8, 0.9, 1, 1.1, or 1.2. In certain embodiments, said reduction is achieved within twelve weeks after first administering the pharmaceutical composition. In certain embodiments, said reduction is achieved within 4, 5, 6, 7, 8, 9 or 10 weeks after first administering the pharmaceutical composition.

In certain embodiments, after a duration of at least three months where the patient has received a dose of pharmaceutical composition each day, the patient has a vascular engorgement score of no greater than 2. In certain embodiments, after a duration of at least three months where the patient has received a dose of pharmaceutical composition each day, the patient has a vascular engorgement score of no greater than 1.5. In certain embodiments, after a duration of at least three months where the patient has received a dose of pharmaceutical composition each day, the patient has a vascular engorgement score of no greater than 1. In certain embodiments, after a duration of at least three months where the patient has received a dose of pharmaceutical composition each day, the patient has a vascular engorgement score of 0. In certain embodiments, after a duration of at least three months where the patient has received a dose of minocycline topical suspension each day, the patient has a vascular engorgement score of no greater than 2.0, 1.9, 1.8, 1.7, 1.6, 1.5, 1.4, 1.3, 1.2, 1.1, 1.0, 0.9, 0.8, or 0.7.

### Reduction in Eye Discomfort Visual Analog Score

The methods may be further characterized according to the reduction in the patient's eye discomfort visual analog score. For example, in certain embodiments, the method produces a reduction in Eye Discomfort Visual Analog Score of at least 20 percent. In certain embodiments, said reduction is achieved within twelve weeks after first administering the pharmaceutical composition. In certain embodiments, the method produces a reduction in Eye Discomfort Visual Analog Score of at least 20, 21, 22, 23, 24, 25, 26, 27, 28, 29, 30, 31, 32, 33, 34, or 35 percent. In certain embodiments, said reduction is achieved within 4, 5, 6, 7, 8, 9 or 10 weeks after first administering the pharmaceutical composition.

In certain embodiments, after a duration of at least three months where the patient has received a dose of pharmaceutical composition each day, the patient has an Eye Discomfort Visual Analog Score of no greater than 50. In certain embodiments, after a duration of at least three months where the patient has received a dose of pharmaceutical composition each day, the patient has an Eye Discomfort Visual Analog Score of no greater than 40. In certain embodiments, after a duration of at least three months where the patient has received a dose of pharmaceutical composition each day, the patient has an Eye Discomfort Visual Analog Score of no greater than 30. In certain embodiments, after a duration of at least three months where the patient has received a dose of pharmaceutical composition each day, the patient has an Eye Discomfort Visual Analog Score of no greater than 25. In certain embodiments, after a duration of at least three months where the patient has received a dose of pharmaceutical composition each day, the patient has an Eye Discomfort Visual Analog Score of no greater than 10. In certain embodiments, after a duration of at least three months where the patient has received a dose of minocycline topical suspension each day, the patient has an Eye Discomfort Visual Analog Score of no greater than 50, 45, 40, 39, 38, 37, 36, 35, 34, 33, 32, 31, or 30.

### Tear Osmolarity

The method may be further characterized according to the patient's tear osmolarity after receiving the pharmaceutical composition. For example, in certain embodiments, the method produces a reduction in tear osmolarity value in the patient. For example, in certain embodiments, the patient's tear osmolarity value is reduced to less than about 310, 312, or 315 mOsmol/L. In certain embodiments, the patient's tear osmolarity value is reduced to about 308 mOsmol/L.

In certain embodiments, the method produces at least a 1%, 2%, 3%, 4%, 5%, 6%, 7%, 8%, 9%, 10%, 12%, 14%, 16%, 18%, or 20% reduction in tear osmolarity value in the patient compared to the patient's tear osmolarity value prior to starting treatment using the pharmaceutical composition. In certain embodiments, the method produces at least a 1%, 2%, 3%, or 4% reduction in tear osmolarity value in the patient compared to the patient's tear osmolarity value prior to starting treatment using the pharmaceutical composition. In certain embodiments, the method produces at least a 1% to 5% reduction in tear osmolarity value in the patient compared to the patient's tear osmolarity value prior to starting treatment using the pharmaceutical composition. In certain embodiments, the method produces a reduction in tear osmolarity value in the range of from about 5% to about 10%, from about 10% to about 20%, or from about 20% to about 50% in the patient compared to the patient's tear osmolarity value prior to starting treatment using the pharmaceutical composition.

In certain embodiments, said reduction is achieved within twelve weeks after first administering the pharmaceutical composition. In certain embodiments, said reduction is achieved within 4, 5, 6, 7, 8, 9 or 10 weeks after first administering the pharmaceutical composition.

### Tear Film MMP-9 Concentration

The methods may be further characterized according to the patient's tear film MMP-9 concentration after receiving the pharmaceutical composition. For example, in certain embodiments, the method produces a reduction in MMP-9 concentration in the patient's tear film. For example, in certain embodiments, the patient's tear film MMP-9 concentration is reduced to less than about 5, 10, 15, 20, 25, 30, 35, or 40 ng/mL. In certain embodiments, the patient's tear film MMP-9 concentration is reduced to less than about 20 ng/mL. In certain embodiments, the patient's tear film MMP-9 concentration is reduced to less than about 40 ng/mL. In certain embodiments, the patient's tear film MMP-9 concentration is reduced to a concentration in the range of from about 3 ng/mL to about 40 ng/mL. In certain embodiments, the patient's tear film MMP-9 concentration is reduced to a concentration in the range of from about 3 ng/mL to about 20 ng/mL. In certain embodiments, the patient's tear film MMP-9 concentration is reduced to a concentration in the range of from about 20 ng/mL to about 40 ng/mL.

In certain embodiments, the method produces at least a 1%, 2%, 3%, 4%, 5%, 6%, 7%, 8%, 9%, 10%, 12%, 14%, 16%, 18%, 20%, 30%, 40%, 50%, 60%, 70%, 80%, 90%, or 100% reduction in MMP-9 concentration in the patient's tear film compared to the patient's tear film MMP-9 concentration prior to starting treatment using the pharmaceutical composition. In certain embodiments, the method produces at least a 5% reduction in MMP-9 concentration in the patient's tear film compared to the patient's tear film MMP-9 concentration prior to starting treatment using the pharmaceutical composition. In certain embodiments, the method produces at least a 10% reduction in MMP-9 concentration in the patient's tear film compared to the patient's tear film MMP-9 concentration prior to starting treatment using the pharmaceutical composition. In certain embodiments, the method produces at least a 20% reduction in MMP-9 concentration in the patient's tear film compared to the patient's tear film MMP-9 concentration prior to starting treatment using the pharmaceutical composition.

In certain embodiments, said reduction is achieved within twelve weeks after first administering the pharmaceutical composition. In certain embodiments, said reduction is achieved within 4, 5, 6, 7, 8, 9 or 10 weeks after first administering the pharmaceutical composition.

### Tear Film Breakup Time

The methods may be further characterized according to the patient's tear film breakup time after receiving the pharmaceutical composition. For example, in certain embodiments, the method produces an increase in tear film breakup time. For example, in certain embodiments, the tear film break up time is increased to at least 10, 12, 14, or 16 seconds. In certain embodiments, the tear film break up time is increased to at least 10 seconds. In certain embodiments, the tear film break up time is increased to within the range of 10 seconds to 20 seconds. In certain embodiments, the tear film break up time is increased to within the range of 10 seconds to 14 seconds.

In certain embodiments, the method produces at least a 1%, 2%, 3%, 4%, 5%, 6%, 7%, 8%, 9%, 10%, 12%, 14%, 16%, 18%, 20%, 30%, 40%, 50%, 60%, 70%, 80%, 90%, or 100% increase in tear film breakup time compared to the patient's tear film break up time prior to starting treatment using the pharmaceutical composition. In certain embodiments, the method produces at least a 10% increase in tear film breakup time compared to the patient's tear film break up time prior to starting treatment using the pharmaceutical composition. In certain embodiments, the method produces at least a 25% increase in tear film breakup time compared to the patient's tear film break up time prior to starting treatment using the pharmaceutical composition. In certain embodiments, the method produces at least a 50% increase in tear film breakup time compared to the patient's tear film break up time prior to starting treatment using the pharmaceutical composition.

In certain embodiments, said increase is achieved within twelve weeks after first administering the pharmaceutical composition. In certain embodiments, said increase is achieved within 4, 5, 6, 7, 8, 9 or 10 weeks after first administering the pharmaceutical composition.

### O. Compositions for Medical Use

Pharmaceutical compositions described herein may be used to treat a medical condition described herein. The use may be according to a method described herein. For example, one aspect of the invention provides a pharmaceutical composition for use in treating meibomian gland dysfunction, wherein the use consists of topically administering to the eyelid margin of a patient in need thereof a therapeutically effective amount of a pharmaceutical composition to treat the meibomian gland dysfunction, wherein the pharmaceutical composition consists of:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

Embodiments described herein in connection with the methods for treatment may be applied in connection with the pharmaceutical composition for use.

### P. Preparation of a Medicament

Pharmaceutical compositions described herein may be used in the preparation of a medicament to treat a medical condition described herein. For example, one aspect of the invention provides for the use of a pharmaceutical composition described herein in the preparation of a medicament for treating meibomian gland dysfunction, wherein the use consists of topically administering to the eyelid margin of a patient in need thereof a therapeutically effective amount of a pharmaceutical composition to treat the meibomian gland dysfunction, wherein the pharmaceutical composition and/or medicament consists of:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

Embodiments described herein in connection with the methods for treatment may be applied in connection with the pharmaceutical composition for use in the preparation of a medicament.

### Q. Medical Kits

Another aspect of the invention provides a medical kit comprising, for example, (i) a composition described herein, and (ii) instructions for treating meibomian gland dysfunction according to methods described herein.

Another aspect of the invention provides a medical kit comprising, for example, (i) a composition described herein, and (ii) instructions for treating dry eye disease according to methods described herein.

### Examples

The invention now being generally described, will be more readily understood by reference to the following examples, which are included merely for purposes of illustrating certain aspects and embodiments of the present invention, and are not intended to limit the invention.

### Example 1—Pharmaceutical Composition Preparation

A pharmaceutical composition for use in topical application to patients was prepared according to the procedures described below.

General Procedure: Mineral oil was mixed with the polymeric hydrocarbon gelling agent sold under the tradename VERSAGEL® M-750, which is a mixture of ethylene-propylene-styrene copolymer, butylene-ethylene-styrene copolymer, butylated-hydroxytoluene, and mineral oil. The polymeric hydrocarbon gelling agent sold under the tradename VERSAGEL® M-750 contained (i) ethylene-propylene-styrene copolymer (having a weight-average molecular weight of about 200,000 g/mol) in an amount within the range of 2.5% to 10% (w/w), (ii) butylene-ethylene-styrene copolymer (having a weight-average molecular weight of about 100,000 g/mol) in an amount within the range of 0.1% to 2.5% (w/w), (iii) butylated-hydroxytoluene in an amount <0.5% (w/w), and (iv) mineral oil (e.g., having a weight-average molecular weight in the range of 230-700 g/mol). A description of the pharmaceutical composition prepared is described in Table 1 below.

TABLE 1
Pharmaceutical Composition

| Component | Amount |
|---|---|
| Polymeric hydrocarbon gelling agent | 70% w/w |
| commercially available under the | |
| tradename VERSAGEL ® M-750 | |
| Mineral oil | 30% w/w |

The pharmaceutical composition was subjected to sterilization, and then packaged in lacquer-lined aluminum tubes with a nasal tip and a low density polyethylene cap closure.

### Example 2—Treatment of Meibomian Gland Dysfunction in Human Subjects

In a clinical study, human subjects suffering from meibomian gland dysfunction were treated by topical administration of a Test Pharmaceutical Composition to the eyelid margin of the subject. Subjects were evaluated for improvement. Experimental procedures for the clinical study are provided below, along with results from the clinical study.

### Part I—Experimental Procedures

### A. Study Summary

Human subjects suffering from inflamed meibomian gland dysfunction that met enrollment criteria were enrolled in the study. Test Pharmaceutical Composition (also referred to as Investigational Product (IP)) was administered to both eyes twice daily (i.e., BID) during the study. Each dose of IP was delivered using a fingertip, as an instillation of approximately ¼ inch strip (equivalent to approximately 50 μL drop) in each eye. IP was instilled to full eyelid margin. Subjects were instructed to wash hands thoroughly prior to administration of IP. Following administration of the IP, subjects were allowed to blot or clean the lower eyelid skin, if necessary.

At Visit 1 (Screening) informed consent was obtained from subjects and eligibility was determined. After the screening assessment, eligible subjects entered into a 2-week run-in period, during which IP BID was instilled in each eye.

At Visit 2 (Baseline) eligibility was reconfirmed and subjects began daily BID administration of IP for twelve weeks. At Visit 3 (Week 2), Visit 4 (Week 4), Visit 5 (Week 8), and Visit 6 (Week 12) subjects attend clinic visits where efficacy and safety evaluations were performed. Subjects who discontinued before Visit 6 underwent Visit 6 evaluations (at the Early Termination visit).

At Visit 7 (Week 16) Post-Treatment Follow-up Visit, efficacy and safety evaluations were performed.

### B. Investigational Product (IP)

IP was supplied as a sterile ointment packaged in a 5 gm lacquer-lined aluminum tube with a nasal tip and a low density polyethylene cap closure. Composition of the IP is set forth in Table 2.

TABLE 2
Composition of Investigational product (IP)

| Component | Amount |
|---|---|
| Polymeric hydrocarbon gelling agent | 70% w/w |
| commercially available under the | |
| tradename VERSAGEL ® M-750 | |
| Mineral oil | 30% w/w |

The polymeric hydrocarbon gelling agent commercially available under the tradename VERSAGEL® M-750 is a mixture of ethylene-propylene-styrene copolymer, butylene-ethylene-styrene copolymer, butylated-hydroxytoluene, and mineral oil, wherein the ethylene-propylene-styrene copolymer (having a weight-average molecular weight of about 200,000 g/mol) was present in an amount within the range of 2.5% to 10% (w/w), the butylene-ethylene-styrene copolymer (having a weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol). The IP was prepared based on procedures described in Example 1, whereby the polymeric hydrocarbon gelling agent commercially available under the tradename VERSAGEL® M-750 was mixed with mineral oil (USP Mineral Oil, having a specific gravity from 0.845 to 0.905 and a kinematic viscosity of not less than 34.5 centistokes at 40° C.) and the resulting mixture subjected to sterilization to produce IP having the composition set forth in Table 2.

### C. IP Dispensation Instructions

At Visit 1, (Day −14/Screening), subjects received their first dose of IP which was self-administered in the clinic under the supervision of the Dedicated Dosing Coordinator. IP was instilled to the full eyelid margin. Subjects were instructed to wash hands thoroughly prior to administration of IP. (Following administration of the IP, subjects were allowed to blot or clean the lower eyelid skin, if necessary.)

They then received one tube of IP from a general supply to take home for self-administration. The tube of IP was returned to the site at Visit 2 (Day 1).

At Visit 2, subjects were assigned a kit containing six tubes of IP. The first dose of IP was self-administered in the clinic by the subject under the supervision of the Dedicated Dosing Coordinator. Subjects then received one tube of IP from their assigned kit to take home for self-administration. Subjects were instructed to apply IP to the full eyelid margin and to wash hands thoroughly prior to administration of IP as was the same at Visit 1.

Subjects were instructed to return the used tube of IP at Visit 3 and at Visit 3 will receive one new tube of IP which is returned at Visit 4. At Visits 4 and 5, subjects received two new tubes of IP at each visit which were returned at Visits 5 and 6, respectively.

### D. Timing of Self-Administration

At Visit 1, subjects self-administer the first dose of IP in the clinic under the supervision of the Dedicated Dosing Coordinator. Subjects then self-administer additional doses of investigational product during the remainder of the run-in period. At Visit 2, subjects self-administer their first dose of IP under the supervision of the Dedicated Dosing Coordinator. Subjects then self-administer additional doses of IP during the remainder of the study. Visits were to be scheduled in the morning to allow subjects to receive the two daily doses 8 to 12 hours apart, with the AM dose occurring in the clinic for Visit 2/Day 1±2 days.

Subjects were asked to instill the first daily dose upon awakening and then the second daily dose approximately 8 to 12 hours later. The two daily doses are described as “Morning (AM) Dose” and “Evening (PM) Dose.”

Following Visit 2, clinic visits were scheduled prior to the subject administration of their morning dose if possible. If the subject did take a dose, the visit was to be scheduled at least 2 hours following the morning dose to prevent the subject from being evaluated with residual investigational product on the eyelids.

### E. Efficacy Endpoints

### 1. Primary Efficacy Endpoints

The following primary efficacy endpoints were used to evaluate effect of the IP:
Changes in Vascular Engorgement at the study eyelid margin as graded by the investigator at Week 12 (Visit 6) from Baseline (Visit 2)
Change in Eye Discomfort Visual Analogue Score (VAS) at Week 12 (Visit 6) from Baseline (Visit 2)

### 2. Secondary Efficacy Endpoints

Secondary endpoints of the study include:
Change in Fluorescein Corneal Staining (FCS) total score (NEI/Industry Workshop 0-15 scale) in the study eye at Week 4 (Visit 4), Week 8 (Visit 5), Week 12 (Visit 6), and Week 16 (Visit 7) from Baseline (Visit 2).
Change in FCS inferior, nasal, and central combined score (NEI/Industry Workshop section 1, 4 and 5 with a 0-9 scale) in the study eye at Week 4 (Visit 4), Week 8 (Visit 5), Week 12 (Visit 6), and Week 16 (Visit 7) from Baseline (Visit 2).
Change in FCS inferior score (NEI/Industry Workshop section 5 with a 0-3 scale) in the study eye at Week 4 (Visit 4), Week 8 (Visit 5), Week 12 (Visit 6), and Week 16 (Visit 7) from Baseline (Visit 2).
Changes in Eye Discomfort Visual Analogue Score (VAS) at Week 4 (Visit 4), Week 8 (Visit 5), Week 12 (Visit 6), and Week 16 (Visit 7) from Baseline (Visit 2).
Changes in Investigator-rated assessments of objective signs of meibomian gland dysfunction (MGD) including change from Baseline (Visit 2) at each follow up visit using individual severity scores:
   Vascular engorgement of the eyelid margin
   Plugging of Meibomian Gland
   Character of Secretion Expressed
   Expressibility of the Meibomian Gland
Changes in Investigator reported scores on objective signs of Meibomian Gland Dysfunction for Total Clinical Outcome Severity Score from Baseline (Visit 2) defined as the sum of the four individual severity scores for the clinical signs of:
   Vascular Engorgement of eyelid margin
   Plugging of Meibomian Gland
   Character of Secretion Expressed
   Expressibility of the Meibomian Gland
Changes in Investigator reported scores on objective Conjunctival Tarsal Erythema change from Baseline (Visit 2) at each follow up visit.
Changes in Tear Film Break-Up Time (TFBUT) from Baseline (Visit 2) at each follow up visit.
Changes in the following Dry Eye-related ocular symptoms at each follow-up visit:
   VAS Scale Symptoms (other than eye discomfort):
      Eye Dryness
      Foreign Body Sensation
   SANDE change in the square root of the product of the two questions at each timepoint compared to baseline and the change from baseline of each individual question within the SANDE
The mean tear osmolarity score at each follow up visit compared to baseline.
The proportion of subjects converting from positive point of care matrix metalloproteinase-9 (MMP-9) to negative point of care MMP-9 at Week 12 (Visit 6).
Number of symptom flares during the 12 weeks of treatment.
The change from baseline in the unanesthetized Schirmer score.

### 3. Safety Endpoints

Safety endpoints of the study include:
Adverse Event (AE) Monitoring
Best Corrected Visual Acuity (BCVA)
Slit Lamp Biomicroscopy and External Eye Exam
Intraocular Pressure (IOP) Measurement
Dilated Ophthalmoscopy
Follow-Up Assessment

### F. Selection of Subjects

### 1. Subject Inclusion Criteria

At Visit 1, individuals of any gender or any race were eligible for study participation if they:
1. Provided written informed consent prior to any study procedures.
2. Were 18 years of age or above.
3. Had a clinical diagnosis of moderate to severe MGD and who meet the following criteria, in a qualifying eyelid, at both Visit 1 (Screening) and Visit 2 examinations:
   a. Clinical sign severity score of at least 2 (moderate) on vascular engorgement at the eyelid margin and
   b. Clinical sign severity score of at least 2 (moderate) on plugging of the meibomian glands.
   c. Eye Discomfort Symptom score of >40 using VAS (0-100 point scale)
4. Met the following criteria, in a qualifying eye (same eye that qualifies for Inclusion #3), at both the Visit 1 (Screening) and Visit 2 examinations:
   a. Fluorescein corneal staining (FCS) total score >3 in the inferior, central, and nasal region combined score (NEI/Industry Workshop sections 1, 4 and 5 with 0-9 scale)
   b. Schirmer score of >7 mm without topical anesthesia
5. Were willing and able to follow instructions and can be present for the required study visits for the duration of the study.
6. Had a BCVA, using corrective lenses if necessary, in both eyes of at least +0.7 as assessed by Early Treatment of Diabetic Retinopathy Study (ETDRS) or modified ETDRS.
7. If female, were non-pregnant, non-lactating and women of childbearing potential (WOCBP) must be using an acceptable method of birth control [e.g., an Intrauterine Contraceptive Device (IUCD) with a failure rate of <1%, hormonal contraceptives, or a barrier method] for the duration of the study. If a female subject was currently abstinent, they agreed to use one of the acceptable methods of birth control before they become sexually active.

### 2. Subject Exclusion Criteria

In order for subjects to be eligible at Visit 1 they may not:
1. Have presence of inflammation and/or active structural change in the iris or anterior chamber.
2. Have lid structural abnormalities such as entropion or ectropion.
3. In the eyelid that qualifies (based on Inclusion #3), have grade level 4 (Obstructed) on Character of Secretion of Meibomian Glands or grade level 4 (No glands are expressible) on the Expressibility of Meibomian Glands.
4. Subjects with ocular inflammatory conditions (e.g., conjunctivitis, keratitis, anterior blepharitis, etc.) not related to MGD.
5. Subjects who have FCS total score=15 or a score=3, in either eye, in the superior region NEI/Industry Workshop scale or subjects who have FCS with diffuse confluent staining, filaments or frank epithelial defects.
6. Have suspected ocular fungal, viral or bacterial infection.
7. Have had penetrating intraocular surgery in the past 90 days or require penetrating intraocular surgery during the study.
8. Have had ocular surface surgery within 12 months of Visit 1 (e.g., LASIK, refractive, pterygium removal).
9. Subjects who within the past 90 days have had cauterization of the punctum or changes to the status (insertion or removal) of punctal plug(s) before the Screening Visit.
10. Have used topical ocular or oral antibiotics within 30 days of the study or expect to use during the study.
11. Have used LipiFlow or hypochlorous acid spray within 30 days of the study or expect to use during the study.
12. If using inhaled or intranasal corticosteroids, unable to maintain a stable dose for the duration of the study.
13. Have ever used isotretinoin.
14. If using Omega-3 supplements, dose must be stable for 3 months prior to Visit 1 and for the duration of the study.
15. Have used topical cyclosporine within 30 days of the study or during the study.
16. Have used topical lifitegrast within 30 days of the study or during the study.
17. Have used systemic corticosteroids within 30 days prior to study entry or during study participation.
18. Have used topical ocular corticosteroids or ocular non-steroidal anti-inflammatory drugs (NSAIDs) within 30 days prior to study entry and during study participation.
19. Have used topical ocular antihistamine and/or mast cell stabilizers within 30 days prior to study entry or during study participation.
20. Are unable or unwilling to discontinue using any preserved or unpreserved topical ocular medications (including artificial tears) upon Screening and for the duration of the study.
21. Are unwilling to discontinue use of contact lenses during the study.
22. Are unwilling to discontinue use of cosmetic makeup applied to the eyelids or eye lashes at the Screening Visit and during the study. If makeup was used, it should be removed at least 12 hours prior to the Visit 1.
23. Have a known hypersensitivity to minocycline, any tetracycline antibiotic, or to ingredients in the investigational product.
24. Are unable or unwilling to withhold the use of eyelid scrubs or use of mechanical therapy during the study.
25. Have been diagnosed with glaucoma or are currently using any glaucoma medication.
26. Have a history of herpetic keratitis.
27. Have a concomitant ocular pathology other than condition under study assessed as potentially confounding by the investigator.
28. Have a serious systemic disease or uncontrolled medical condition that in the judgment of the investigator could confound study assessments or limit compliance.
29. Have been exposed to minocycline, any investigational drug, or investigational device within the preceding 30 days.
30. Are an employee of the site that is directly involved in the management, administration, or support of this study or be an immediate family member of the same.
31. Have trigger factors including conjunctivochalasis, allergic conjunctivitis, contact lens intolerance, trichiasis, epithelial basement membrane dystrophy, infectious keratitis or conjunctivitis
32. Have a documented history of ocular allergies, which, in the judgment of the investigator, are likely to have an acute increase in severity due to the expected timing of the exposure to the allergen to which the subject is sensitive. Subjects sensitive to seasonal allergens that are not expected to be present during the study are permitted.

### 3. Study Eye Selection

The study eye was the eye with the eyelid at Visit 2, having the worst (higher) score defined as the sum of the following two severity scores for the clinical signs of Meibomian Gland Disease (Note, the study eye and eyelid must have met qualifying eligibility criteria at Visit 1 and Visit 2):
1. Vascular Engorgement of eyelid margin
2. Plugging of the Meibomian Gland

If both eyes, and eyelids, had the same score, then the right eye and upper eyelid was selected.

### G. Visit 2

At the Visit 2, an eligible subject must continue to meet all clinical inclusion/exclusion criteria as defined above. Subjects must meet all criteria from Visit 1 and inflamed MGD criteria in the same qualifying eye and/or qualifying eyelid as in Visit 1. Further, subjects must be 80% compliant with respect to dosing of run-in IP and diary completion.

The absence or presence of evaporative DED in the study eye was noted. A clinical diagnosis of evaporative DED in the study eye, was defined as meeting the following criteria at Visit 2 (Baseline):
1. Fluorescein corneal staining (FCS) total score >6 in the inferior, central, and nasal region combined score (NEI/Industry Workshop section 1, 4 and 5 with 0-9 scale) and
2. Symptom Severity score of >50 using the SANDE questionnaire

### H. Visit Descriptions

Visit assessments were performed in the order suggested in both eyes.

### 1. Visit 1 (Screening): 14 (±2) Days Prior to Visit 2

The following was performed/assessed in the order suggested below and in both eyes:
Explain the purpose and conduct of the study to the subject, answer the subject's questions, and obtain written informed consent.
Obtain information including: demographics, concomitant medications, ocular and systemic medical and medication history and surgical history.
A Screening ID is assigned to the Subject once any Visit 1 procedures are performed.
Subject Rated Symptom Assessments (in this order):
   1. Individual Symptom Assessment via VAS
   2. SANDE
For all eligible women of childbearing potential, perform a urine pregnancy test (UPT) to confirm that the subject is not pregnant.
BCVA
Tear Osmolarity (conducted at a subset of sites)
External Eye Exam
Slit-lamp biomicroscopy
Investigator-rated assessment of Meibomian Gland Dysfunction (in both eyes for upper and lower lids)
   See section below. Assessment of Efficacy for full description of investigator-rated assessments for objective signs of change from baseline using five individual severity scores
TFBUT
FCS (NEI/Industry Workshop scale)
Unanesthetized Schirmer test
Wait 10 minutes prior to MMP-9 Assessment
MMP-9 Point of Care Assessment
IOP
Dilated Ophthalmoscopy
Determine if the subject is eligible to continue in the study. Do not continue screening any subject who does not meet eligibility requirements. Any subject who does not meet eligibility requirements are designated as a Screen Failure.
Instruct the subject to discontinue using all ophthalmic medications that he/she had been using before the screening visit. Remind the subject that they are not to use artificial tears or any other OTC or prescription or any other topical eye medication other than the investigational drug they have been given during the remainder of the study.
If the subject is qualified, a three-week supply of IP is dispensed.

### 2. Visit 2: Day 1 (±2 Days)

Visit 2 occurs 14 (±2) days after Visit 1 (Screening). The following was performed/assessed in both eyes:
Subject Rated Symptom Assessments (in this order):
   1. Individual Symptom Assessment via VAS
   2. SANDE
Subject is asked the following question regarding symptom flare experienced the day prior to the visit:
   During the prior day, did you experience any discrete and severe episodes of eye discomfort (related to your MGD) lasting more than a minute, and if so, how many episodes?
Use of any concomitant medications since the last visit
Occurrence of any AEs since the last visit
Compliance is assessed via review of the daily dosing information recorded by the subject. Subjects must be 80% compliant with respect to dosing and diary completion for eligibility.
BCVA
Tear Osmolarity (at a subset of sites)
External Eye Exam
Slit-lamp biomicroscopy
Investigator-rated assessment of signs of Meibomian Gland Dysfunction (in both eyes for upper and lower lids)
   See section below. Assessment of Efficacy for full description of investigator-rated assessments for objective signs of change from baseline using five individual severity scores
TFBUT
FCS (NEI/Industry Workshop scale)
Unanesthetized Schirmer test
IOP

### 3. Visit 3: Day 15 (±2 Days)

This visit occurs on Day 15 as calculated from Visit 2: Day 1, and the following was performed in both eyes:
Subject Rated Symptom Assessments (in this order):
   1. Individual Symptom Assessment via VAS
   2. SANDE
Use of any concomitant medications since the last visit
Occurrence of any AEs since the last visit
BCVA
Tear Osmolarity (at a subset of sites)
External Eye Exam
Slit-lamp biomicroscopy
Investigator-rated assessment of signs of Meibomian Gland Dysfunction
   See section below. Assessment of Efficacy for full description of investigator-rated assessments for objective signs of change from baseline using five individual severity scores
TFBUT
FCS (NEI/Industry Workshop scale)
IOP

### 4. Visit 4: Day 29 (±2 Days)

This visit occurs on Day 29 as calculated from Visit 2: Day 1, and the following was performed in both eyes:
For all eligible women of childbearing potential, perform a urine pregnancy test to confirm that the subject is not pregnant.
Subject Rated Symptom Assessments (in this order):
   1. Individual Symptom Assessment via VAS
   2. SANDE
Use of any concomitant medications since the last visit
Occurrence of any AEs since the last visit
BCVA
Tear Osmolarity (at a subset of sites)
External Eye Exam
Slit-lamp biomicroscopy
Investigator-rated assessment of signs of Meibomian Gland Dysfunction
   See section below. Assessment of Efficacy for full description of investigator-rated assessments for objective signs of change from baseline using five individual severity scores
TFBUT
FCS (NEI/Industry Workshop scale)
IOP

### 5. Visit 5: Day 57 (±2 Days)

This visit occurs on Day 57 as calculated from Visit 2: Day 1, and the following was performed in both eyes:
Subject Rated Symptom Assessments (in this order):
   1. Individual Symptom Assessment via VAS
   2. SANDE
Use of any concomitant medications since the last visit
Occurrence of any AEs since the last visit
BCVA
Tear Osmolarity (at a subset of sites)
External Eye Exam
Slit-lamp biomicroscopy
Investigator-rated assessment of signs of Meibomian Gland Dysfunction
   See section below. Assessment of Efficacy for full description of investigator-rated assessments for objective signs of change from baseline using five individual severity scores
TFBUT
FCS (NEI/Industry Workshop scale)
IOP

### 6. Visit 6 (End of Treatment): Day 85 (±2 Days)

This visit occurs on Day 85 as calculated from Visit 2: Day 1, and the following was performed in both eyes:
UPT
Subject Rated Symptom Assessments (in this order):
   1. Individual Symptom Assessment via VAS
   2. SANDE
Use of any concomitant medications since the last visit
Occurrence of any AEs since the last visit
BCVA
Tear Osmolarity (at a subset of sites)
External Eye Exam
Slit-lamp biomicroscopy
Investigator-rated assessment of signs of Meibomian Gland Dysfunction
   See section below. Assessment of Efficacy for full description of investigator-rated assessments for objective signs of change from baseline using five individual severity scores
TFBUT
FCS (NEI/Industry Workshop scale)
Unanesthetized Schirmer test
Wait 10 minutes prior to MMP-9 point of care test
MMP-9 point of care test
IOP
Dilated Ophthalmoscopy

### 7. Visit 7 (Post-Treatment Follow-Up): Day 113 (±2 Days)

This visit occurs on Day 113 as calculated from Visit 6 (End of Treatment), and the following was performed in both eyes:
UPT
Subject Rated Symptom Assessments:
   1. Individual Symptom Assessment via VAS
   2. SANDE
Use of any concomitant medications since the last visit
Occurrence of any AEs since the last visit
BCVA
Tear Osmolarity (at a subset of sites)
External Eye Exam
Slit-lamp biomicroscopy
Investigator-rated assessment of signs of Meibomian Gland Dysfunction
TFBUT
FCS (NEI/Industry Workshop scale)
Unanesthetized Schirmer test
IOP

The subject may be discharged from the study at this visit.

### 8. Unscheduled Visit

Any visits or procedures performed beyond those specified within the protocol were to be documented.

### 9. Early Termination Visit

In the event of termination prior to Visit 6, every attempt was made to ensure that all the following Visit 6 assessments were performed in both eyes at the Early Termination Visit prior to discharge from the study:
Subject Rated Symptom Assessments: SANDE and Individual Symptom Assessment via VAS
Use of any concomitant medications since the last visit
Occurrence of any AEs since the last visit
Used and unused IP collected and compliance assessed via the daily dosing information recorded by the subject
UPT
BCVA
External Eye Exam
Slit lamp biomicroscopy
Investigator-rated assessment of Meibomian Gland Dysfunction
Tear Osmolarity (at a subset of sites)
TFBUT
FCS (NEI/Industry Workshop scale)
Unanesthetized Schirmer test
Wait 10 minutes prior to MMP-9 point of care test
MMP-9 point of care test
IOP measurement
Dilated Ophthalmoscopy

Include subject withdrawal criteria (i.e., terminating investigational product treatment/trial treatment).

I. Subject Withdrawal and/or Discontinuation

Any subject who wished to discontinue IP use or withdraw from participation in the study for any reason was entitled to do so without obligation. The Investigator could also discontinue any subject from investigational product use or from study participation, if deemed necessary.

### J. Assessment of Efficacy

Efficacy assessments included the following:

### 1. Symptom Assessment in Dry Eye (SANDE)

The SANDE questionnaire was assessed at each visit. The subject was asked the following questions regarding the frequency and severity of their dry eye symptoms:

### 2. Individual Symptom Assessments Via VAS

Subjects were asked the following questions regarding their current symptoms (unrelated to study drug instillation) at each visit. The subject is asked to subjectively rate each ocular symptom (OU) by placing a vertical mark on the horizontal line to indicate the level of discomfort. 0 corresponds to “No Symptoms” and 100 corresponds to “Severe Symptoms”
Subject Instructions: Please review the symptoms below. After your review, please rate how your eyes feel for each of the following symptoms by placing a single vertical mark that represents how your symptom feels at this moment.
   3. Patient Reports of MGD Flare (Assessed Via Daily Dosing Diary with Symptom Flare Question)

Subjects were asked to record each day the following information related to administration of investigational product:
Date
Time of Administration
Symptom Exacerbation Question (see below)

Symptom Flare Question: Each evening, the subject was asked to respond to the following question:
During the day, did you experience any discrete and severe episodes of eye discomfort (related to your MGD) lasting more than a minute, and if so, how many episodes?
No=0
Yes, how many (circle number)? 1, 2, 3, 4, More than 4

### 4. Tear Osmolarity Assessment

Tear osmolarity was collected at each visit at a subset of clinical sites. Tear osmolarity is an objective measurement of the salinity and its concentration in an individual's tears. The mean tear osmolarity score is obtained at each visit.

### 5. Fluorescein Corneal Staining (FCS)/National Eye Institute/Industry Workshop Scale

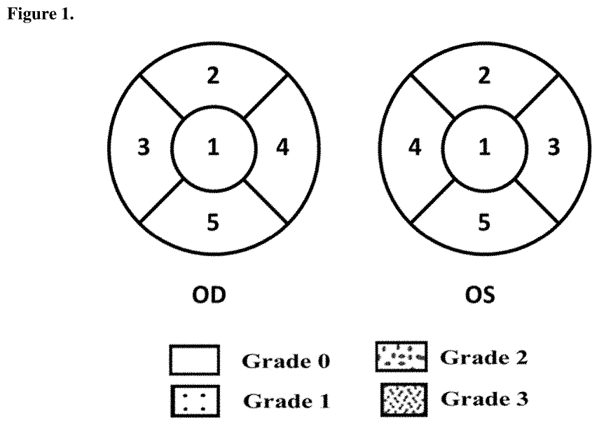

FCS was assessed at each visit. The five areas of the cornea were scored by the investigator according to the scoring system shown in FIG. 1, and the total score is also calculated.

### 6. Unanesthetized Schirmer Test

The Schirmer test was conducted on unanesthetized eyes at Visits 1, 2, 6, and 7. A 35 mm×5 mm filter paper strip was used to measure the amount of tears that are produced over 5 minutes. The strip was placed in the lower eyelid margin without the use of a preplaced ophthalmic anesthetic drop. After 5 minutes, the strip was removed and the amount of wetting is measured in millimeters.

### 7. MMP-9 Point of Care Assessment

The MMP-9 point of care assessment was conducted at Visit 1 and Visit 6. The Quidel InflammaDry test system was used to assess the tear film for the presence of MMP-9. The sample is obtained, and the test performed according to package instructions.

### 8. Tear Film Break-Up Time (TFBUT)

TFBUT was measured at each visit. To measure TFBUT, fluorescein was instilled into the subject's tear film, the subject was allowed to blink once or twice to disperse the fluorescein, and the subject was then asked not to blink while the tear film is observed under a broad beam of cobalt blue illumination using a slit-lamp. The TFBUT is recorded as the number of seconds that elapse between the last blink and the appearance of the first dry spot in the tear film. A TFBUT under 10 seconds is considered abnormal.

9. Investigator-Rated Assessment of Objective Signs Including Change from Baseline for Five Individual Severity Scores

The investigator rated the bilateral severity of the subject's MGD signs at each visit according to the following classification:
1. Vascular engorgement at the eyelid margin (evaluate the entire lid margin and evaluate both upper and low eyelid margins separately)
   (0) Normal
   (1) Mild engorgement
   (2) Moderate engorgement
   (3) Severe engorgement
   (4) Very Severe engorgement
2. Plugging of the meibomian gland (evaluate middle part of upper and lower lid, n=10)
   (0) Normal: Clear orifices of meibomian glands (n=0)
   (1) Mild: Less than ⅓ of orifices plugged but at least one appears to contain turbid or oily secretions (n=1-3)
   (2) Moderate: Between ⅓ and ⅔ of orifices plugged (n=4-6)
   (3) Severe: More than ⅔ of orifices plugged (n=7-9)
   (4) Very severe: All orifices plugged (n=10)
3. Character of secretion expressed from the meibomian gland (evaluate middle part of lower lid, n=10 and middle part of upper lid, n=10))
   (0) Normal: minimal clear secretion
   (1) Mild: cloudy
   (2) Moderate: granular
   (3) Severe: paste
   (4) Obstructed: no observable expressate
4. Expressibility of Meibomian Glands (upper and lower lid out of 10 glands)
   (0) All glands expressible
   (1) At least 5 glands expressible
   (2) Only 3-4 glands expressible
   (3) Only 1-2 glands expressible
   (4) No glands are expressible
5. Conjunctival Tarsal Erythema (evaluate upper and lower tarsal conjunctiva) (Note: Level of severity is not required for eligibility)
   (0) Normal: normal age appropriate vascularity of the tarsal conjunctiva
   (1) Trace erythema: slightly dilated blood vessels; vessels colored pink
   (2) Mild erythema: dilated vessels, color light red
   (3) Moderate erythema: diffuse dilated vessels, bright red in color
   (4) Severe erythema: diffuse dilated vessels, deep red color
      10. Total Clinical Outcome Severity Score Defined as the Sum of the Four Severity Scores for the Clinical Signs of Meibomian Gland Disease, as Determined Above in “9. Investigator-Rated Assessment of Objective Signs Including Change from Baseline for Five Individual Severity Scores”:
1. Vascular Engorgement of eyelid margin
2. Plugging of the Meibomian Gland
3. Character of Secretion Expressed from the Meibomian Gland
4. Expressibility of the Meibomian Gland

### O. Assessment of Safety

Safety parameters included:

### 1. Adverse Event Monitoring

See Section O. Adverse Event Definitions

### 2. ETDRS Best Corrected Visual Acuity

BCVA was conducted at each visit. Visual acuity testing should precede any examination requiring contact with the eye or instillation of study dyes, as is detailed in the order of assessments for each Visit in Section 5.1. Logarithm of the Minimal Angle of Resolution (Log MAR) visual acuity must be assessed using an ETDRS or modified ETDRS chart. Visual acuity testing is performed with best correction using subject's own corrective lenses (spectacles only) or pinhole refraction.

An ETDRS or modified ETDRS chart may be used. If a Lighthouse chart is used (24.5″ by 25″; either reflectance or retro-illuminated), the subject must view the chart from a distance of exactly 4 meters (13.1 feet). If smaller reproductions (18″ by 18″, e.g., Prevent Blindness) are used, the subject viewing distance is exactly 10 feet. Reflectance wall charts are frontally illuminated (60 watt bulb or a well-lit room).

The subject is positioned according to the elevation of the chart (either seated or standing) so that the chart is at a comfortable viewing angle. The right eye is tested first. The subject should attempt to read each letter, line-by-line, left to right, beginning with line 1 at the top of the chart. The subject is told that the chart has letters only, no numbers. If the subject reads a number, he or she is reminded that the chart contains no numbers, and the examiner should then request a letter instead of the number. The subject is asked to read slowly, about 1 letter per second, to achieve the best identification of each letter. He/she is not to proceed to the next letter until he/she has given a definite response. If the subject changes a response before he has read aloud the next letter, then the change must be accepted.

Maximum effort is made to identify each letter on the chart; the subject is encouraged to guess. When it becomes evident that no further meaningful readings can be made, the examiner should stop the test. The number of letters missed or read incorrectly is noted.

In order to provide standardized and well-controlled assessments of visual acuity during the study, the same lighting conditions must be used consistently throughout the study.
Calculations: log MAR VA=Baseline value+(n×0.02)
where: the baseline value is the log MAR number of the last line read (at least 1 letter read correctly in this line), and “n” is the total number of letters missed up to and including the last line read, and “0.02” is the value for each letter

### 3. Slit Lamp Biomicroscopy and External Eye Exam

The biomicroscopy exam was performed at each visit. It was performed with the slit lamp using a beam width and intensity that provide optimal evaluation of the anterior segment. This procedure is performed in the same manner for all subjects observed at the Investigator's site.
Lashes
   0=Normal
   1=Abnormal
Eyelid
   Edema
      0=Normal, no swelling of the lid tissue
      1=Abnormal
Conjunctiva
   Edema
      0=Normal, no swelling of the conjunctiva
      1=Abnormal
Palpebral Conjunctival Erythema
   0=Normal, no redness of the conjunctiva
   1=Abnormal
Cornea
   Infiltrates
      0=Absent
      1=Present
   Endothelial Changes
      0=Normal, None
      1=Abnormal, pigment, keratoprecipitates, guttata
   Edema
      0=Normal None, transparent and clear
      1=Abnormal
Anterior Chamber
   Cells
      0=Normal, No cells seen
      1=Abnormal (+ to +++ cells)
   Flare
      0=Normal, No Tyndall effect
      1=Abnormal, Tyndall beam in the anterior chamber
Lens Pathology
   0=Normal; no opacity in the lens
   1=Abnormal; existing opacity in the lens; aphakic or pseudophakic eyes or other abnormal findings.
Sclera
   Injection
      0=Normal, without any redness
      1=Abnormal

### 4. IOP Measurement

IOP measurements were performed utilizing Goldmann applanation tonometry according to the Investigator's standard procedure. All pressures are recorded in mmHg.

### 5. Dilated Ophthalmoscopy

Dilated ophthalmoscopy includes assessment of the optic nerve head for pallor and cupping (cup to disc ratio), and was performed at Visit 1 and Visit 6. After the ophthalmoscopy procedure, the Investigator determined if findings are within normal limits or are abnormal. For abnormal findings at Visit 1, the Investigator determined whether or not the abnormality would exclude subject from study participation.

### Part II—Results

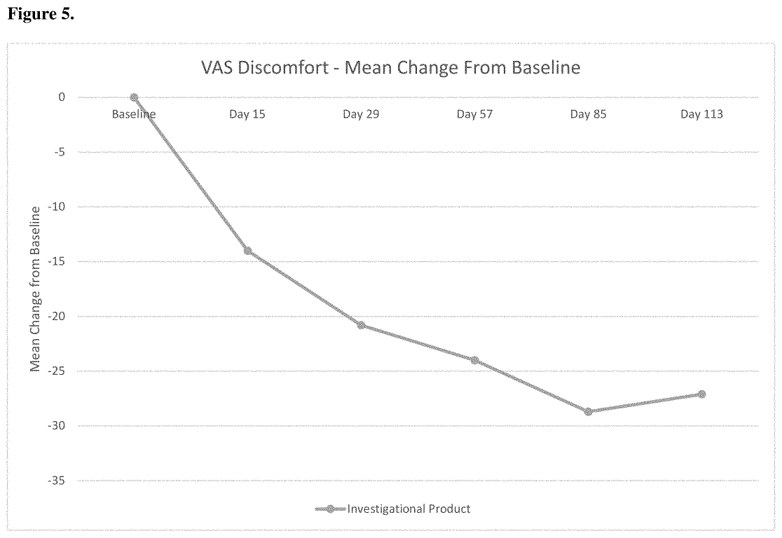

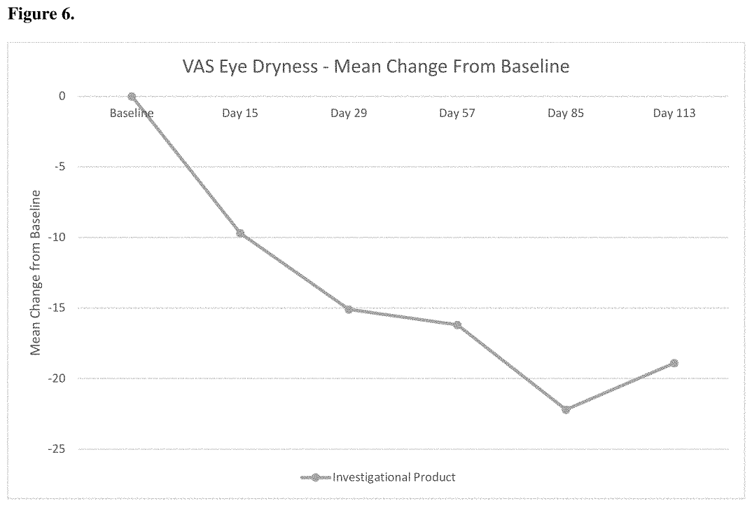

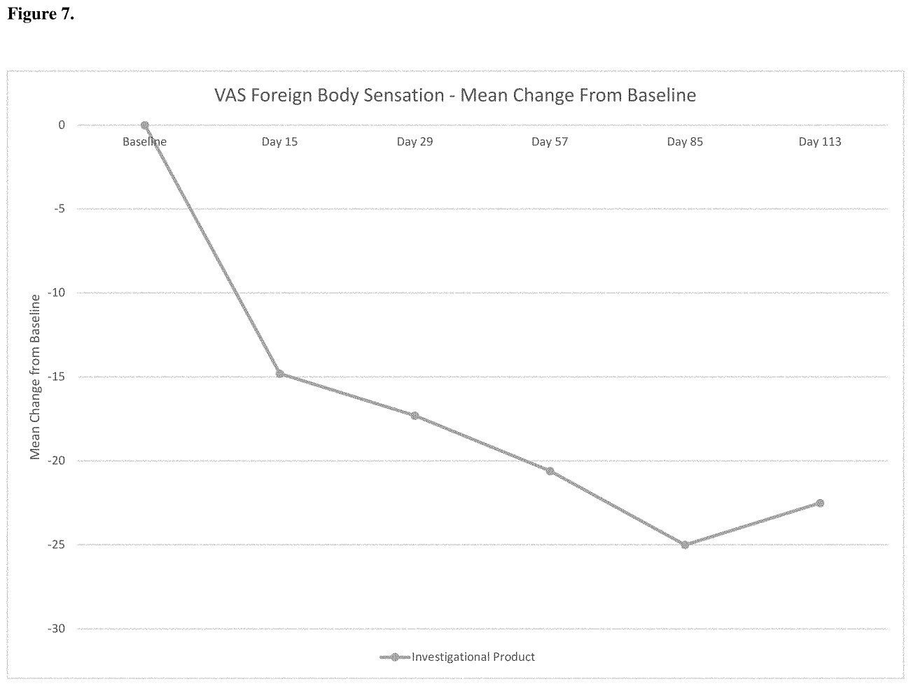

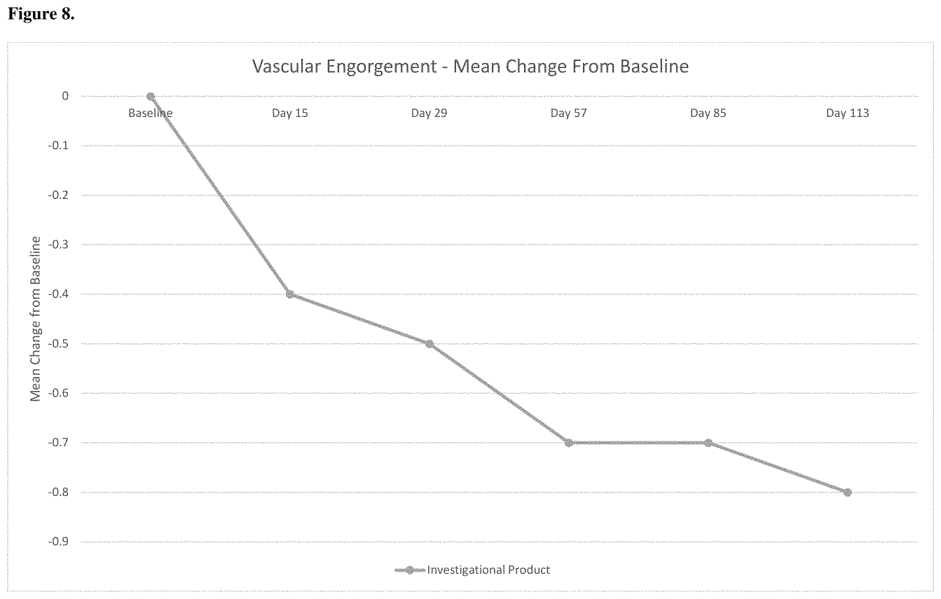

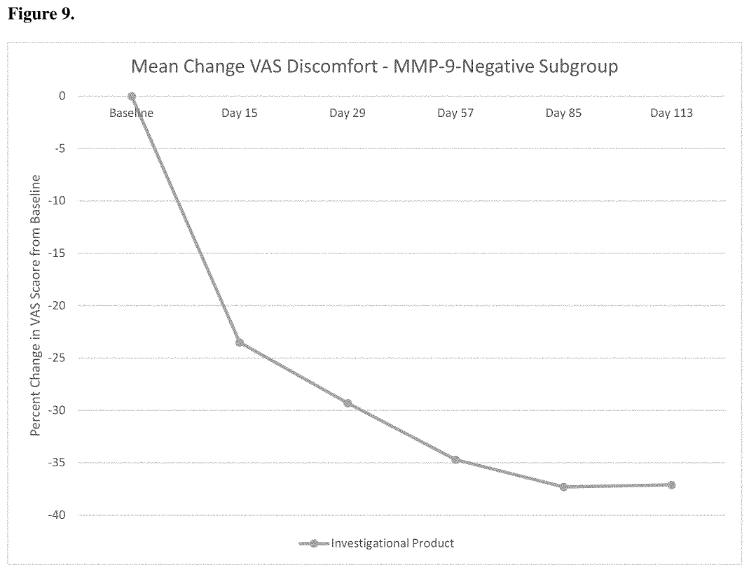

Results of the clinical study are provided in Tables 3-21 below, along with FIGS. 5-9. The results are from 90 subjects enrolled in the study that received IP. Experimental results in Table 3 demonstrate that the Investigational Product was well tolerated by subjects. Experimental results in Tables 4-6 demonstrate improvement in VAS Discomfort, VAS Eye Dryness, and VAS Foreign Body Sensation, respectively, due to the IP.

TABLE 3
Summary of Subjects that Completed vs. Discontinued the Study

| | Number of | |
|---|---|---|
| Subjects | Subjects | Percentage |
| Subjects Who Completed | 79 | 87.8 |
| the Study | | |
| Subjects Who Discontinued | 11 | 12.2 |
| the Study | | |
| Adverse Event | 5 | 5.6 |
| Withdrawn by Subject | 3 | 3.3 |
| Lost to Follow-Up | 2 | 2.2 |
| Other | 1 | 1.1 |

TABLE 4
VAS Discomfort.

| | Mean VAS | Mean Change |
|---|---|---|
| Timepoint | Score | From Baseline |
| Baseline | 65.9 | 0 |
| Day 15 | 53.5 | −14 |
| Day 29 | 44.4 | −20.8 |
| Day 57 | 41.4 | −24 |
| Day 85 | 35.8 | −28.7 |
| Day 113 | 37.7 | −27.1 |

TABLE 5
VAS Eye Dryness.

| | Mean VAS | Mean Change |
|---|---|---|
| Timepoint | Score | From Baseline |
| Baseline | 60.1 | 0 |
| Day 15 | 49.7 | −9.7 |
| Day 29 | 44 | −15.1 |
| Day 57 | 43.1 | −16.2 |
| Day 85 | 36.5 | −22.2 |
| Day 113 | 40 | −18.9 |

TABLE 6
VAS Foreign Body Sensation.

| | Mean VAS | Mean Change |
|---|---|---|
| Timepoint | Score | From Baseline |
| Baseline | 59.4 | 0 |
| Day 15 | 43.9 | −14.8 |
| Day 29 | 41.3 | −17.3 |
| Day 57 | 37.9 | −20.6 |
| Day 85 | 33.5 | −25 |
| Day 113 | 35.8 | −22.5 |

TABLE 7
Vascular Engorgement.

| | Mean | Mean Change |
|---|---|---|
| Timepoint | Score | From Baseline |
| Baseline | 2.3 | 0 |
| Day 15 | 1.9 | −0.4 |
| Day 29 | 1.8 | −0.5 |
| Day 57 | 1.6 | −0.7 |
| Day 85 | 1.5 | −0.7 |
| Day 113 | 1.5 | −0.8 |

TABLE 8
Character of Secretion.

| | Mean | Mean Change |
|---|---|---|
| Timepoint | Score | From Baseline |
| Baseline | 1.9 | 0 |
| Day 15 | 1.8 | −0.1 |
| Day 29 | 1.6 | −0.3 |
| Day 57 | 1.5 | −0.4 |
| Day 85 | 1.4 | −0.5 |
| Day 113 | 1.4 | −0.5 |

TABLE 9
Expressibility of the Meibomian Glands.

| | Mean | Mean Change |
|---|---|---|
| Timepoint | Score | From Baseline |
| Baseline | 1.8 | 0 |
| Day 15 | 1.7 | −0.1 |
| Day 29 | 1.5 | −0.2 |
| Day 57 | 1.4 | −0.4 |
| Day 85 | 1.3 | −0.5 |
| Day 113 | 1.2 | −0.5 |

TABLE 10
Plugging of Meibomian Glands.

| | Mean | Mean Change |
|---|---|---|
| Timepoint | Score | From Baseline |
| Baseline | 2.5 | 0 |
| Day 15 | 2.2 | −0.1 |
| Day 29 | 2 | −0.5 |
| Day 57 | 1.9 | −0.6 |
| Day 85 | 1.7 | −0.8 |
| Day 113 | 1.6 | −0.9 |

TABLE 11
Total MG Outcome.

| | Mean | Mean Change |
|---|---|---|
| Timepoint | Score | From Baseline |
| Baseline | 8.4 | 0 |
| Day 15 | 7.5 | −0.9 |
| Day 29 | 6.8 | −1.6 |
| Day 57 | 6.3 | −2.1 |
| Day 85 | 5.9 | −2.4 |
| Day 113 | 5.7 | −2.7 |

TABLE 12
Tarsal Conjunctival Erythema.

| | | Mean Change |
|---|---|---|
| Timepoint | Mean Score | From Baseline |
| Baseline | 2.2 | 0 |
| Day 15 | 2 | −0.2 |
| Day 29 | 1.8 | −0.4 |
| Day 57 | 1.6 | −0.6 |
| Day 85 | 1.5 | −0.7 |
| Day 113 | 1.4 | −0.9 |

TABLE 13
Total FCS.

| | Mean | Mean Change |
|---|---|---|
| Timepoint | Score | From Baseline |
| Baseline | 5.9 | 0 |
| Day 15 | 4.7 | −1.2 |
| Day 29 | 4.2 | −1.4 |
| Day 57 | 4.1 | −1.7 |
| Day 85 | 3.8 | −2 |
| Day 113 | 3.5 | −2.4 |

TABLE 14
Inferior FCS.

| | Mean | Mean Change |
|---|---|---|
| Timepoint | Score | From Baseline |
| Baseline | 1.9 | 0 |
| Day 15 | 1.6 | −0.3 |
| Day 29 | 1.5 | −0.4 |
| Day 57 | 1.5 | −0.4 |
| Day 85 | 1.5 | −0.4 |
| Day 113 | 1.4 | −0.5 |

TABLE 15
Combined FCS.

| | Mean | Mean Change |
|---|---|---|
| Timepoint | Score | From Baseline |
| Baseline | 4.3 | 0 |
| Day 15 | 3.5 | −0.8 |
| Day 29 | 3.1 | −1.2 |
| Day 57 | 3.1 | −1.2 |
| Day 85 | 2.8 | −1.5 |
| Day 113 | 2.5 | −1.8 |

TABLE 16
VAS Discomfort MMP9-Positive Subgroup.

| | | Mean Percentage |
|---|---|---|
| Timepoint | Mean Score | Change From Baseline |
| Baseline | 65.3 | 0 |
| Day 15 | 54.8 | −16.08% |
| Day 29 | 47.5 | −27.26% |
| Day 57 | 45.6 | −30.17% |
| Day 85 | 38.9 | −40.43% |
| Day 113 | 41.2 | −36.91% |

TABLE 17
Vascular Engorgement MMP9-Positive Subgroup.

| | | Mean Percentage |
|---|---|---|
| Timepoint | Mean Score | Change From Baseline |
| Baseline | 2.3 | 0 |
| Day 15 | 1.9 | −17.39% |
| Day 29 | 1.8 | −21.74% |
| Day 57 | 1.6 | −30.43% |
| Day 85 | 1.5 | −34.78% |
| Day 113 | 1.5 | −34.78% |

TABLE 18
FCS Combined Score MMP9-Positive Subgroup.

| | | Mean Percentage |
|---|---|---|
| Timepoint | Mean Score | Change From Baseline |
| Baseline | 4.2 | 0 |
| Day 15 | 3.4 | −19.05% |
| Day 29 | 3.2 | −23.81% |
| Day 57 | 3.4 | −19.05% |
| Day 85 | 3 | −28.57% |
| Day 113 | 2.6 | −38.10% |

TABLE 19
FCS Inferior Score MMP9-Positive Subgroup.

| | | Mean Percent |
|---|---|---|
| Timepoint | Mean Score | Change from Baseline |
| Baseline | 1.8 | 0 |
| Day 15 | 1.6 | −11.11% |
| Day 29 | 1.6 | −11.11% |
| Day 57 | 1.7 | −5.56% |
| Day 85 | 1.5 | −16.67% |
| Day 113 | 1.4 | −22.22% |

TABLE 20
MG Character of Secretion DED-Positive Subgroup.

| | Mean Character of | Change in Mean Character of |
|---|---|---|
| Timepoint | Secretion Score | Secretion Score Relative to Baseline |
| Baseline | 2.2 | 0 |
| Day 15 | 2 | −0.2 |
| Day 29 | 1.8 | −0.4 |
| Day 57 | 1.6 | −0.6 |
| Day 85 | 1.6 | −0.6 |
| Day 113 | 1.6 | −0.6 |

TABLE 21
Total MGD Score DED-Positive Subgroup.

| | Total MGD | Change in Total MGD |
|---|---|---|
| Timepoint | Score | Score from Baseline |
| Baseline | 9.2 | 0 |
| Day 15 | 8.1 | −1.1 |
| Day 29 | 7 | −2.2 |
| Day 57 | 6.6 | −2.6 |
| Day 85 | 6.8 | −2.4 |
| Day 113 | 6.3 | −2.9 |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating meibomian gland dysfunction, consisting of topically administering to the eyelid margin of a patient in need thereof a therapeutically effective amount of a pharmaceutical composition to treat the meibomian gland dysfunction, wherein the pharmaceutical composition consists of:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

2. The method of claim 1, wherein the meibomian gland dysfunction is mild meibomian gland dysfunction.

3. The method of claim 1, wherein the meibomian gland dysfunction is moderate meibomian gland dysfunction.

4. The method of claim 1, wherein the meibomian gland dysfunction is non-inflammatory meibomian gland dysfunction.

5-11. (canceled)

12. The method of claim 1, wherein the method produces at least a 25% reduction in the average number of symptom flares per month due to meibomian gland dysfunction compared to the average number of symptom flares due to meibomian gland dysfunction experienced by the patient in the month prior to first administering the pharmaceutical composition.

13. (canceled)

14. A method of treating dry eye disease, consisting of topically administering to the eyelid margin of a patient in need thereof a therapeutically effective amount of a pharmaceutical composition to treat the dry eye disease, wherein the pharmaceutical composition consists of:
(a) an ethylene-propylene-styrene copolymer;
(b) optionally a butylene-ethylene-styrene copolymer;
(c) optionally mineral oil;
(d) optionally an antioxidant; and
(e) optionally one or more pharmaceutically acceptable carriers and/or excipients.

15. The method of claim 14, wherein the dry eye disease is aqueous tear deficiency dry eye disease.

16. The method of claim 14, wherein the dry eye disease is evaporative dry eye disease.

17. (canceled)

18. The method of claim 1, wherein ethylene-propylene-styrene copolymer is present in the pharmaceutical composition in an amount of from about 0.1% (w/w) to about 10% (w/w) of the pharmaceutical composition.

19. The method of claim 1, wherein ethylene-propylene-styrene copolymer is present in the pharmaceutical composition in an amount of from about 1.75% (w/w) to about 7% (w/w) of the pharmaceutical composition.

20. (canceled)

21. The method of claim 1, wherein butylene-ethylene-styrene copolymer is present in the pharmaceutical composition.

22. The method of claim 1, wherein butylene-ethylene-styrene copolymer is present in the pharmaceutical composition in an amount of from about 0.01% (w/w) to about 3% (w/w) of the pharmaceutical composition.

23-27. (canceled)

28. The method of claim 1, wherein mineral oil is present in the pharmaceutical composition.

29. The method of claim 1, wherein mineral oil is present in the pharmaceutical composition in an amount of at least 90% (w/w) of the pharmaceutical composition.

30-39. (canceled)

40. The method of claim 1, wherein the pharmaceutical composition consists of:
(a) an ethylene-propylene-styrene copolymer;
(b) a butylene-ethylene-styrene copolymer;
(c) mineral oil;
(d) an antioxidant; and
(e) optionally one or more pharmaceutically acceptable carriers.

41. The method of claim 1, wherein the pharmaceutical composition consists of:
(a) from about 1.75% (w/w) to about 7% (w/w) of an ethylene-propylene-styrene copolymer;
(b) from about 0.07% (w/w) to about 1.75% (w/w) of a butylene-ethylene-styrene copolymer;
(c) mineral oil;
(d) an antioxidant; and
(e) optionally one or more pharmaceutically acceptable carriers.

42. (canceled)

43. The method of claim 1, wherein the pharmaceutical composition consists of:
(a) from about 1.75% (w/w) to about 7% (w/w) of an ethylene-propylene-styrene copolymer;
(b) from about 0.07% (w/w) to about 1.75% (w/w) of a butylene-ethylene-styrene copolymer;
(c) mineral oil; and
(d) an antioxidant.

44. (canceled)

45. The method of claim 40, wherein the ethylene-propylene-styrene copolymer has a weight-average molecular weight in the range of from about 150,000 g/mol to about 250,000 g/mol.

46. (canceled)

47. The method of claim 40, wherein the butylene-ethylene-styrene copolymer has a weight-average molecular weight in the range of from about 50,000 g/mol to about 150,000 g/mol.

48. (canceled)

49. (canceled)

50. The method of claim 40, wherein the mineral oil has a weight-average molecular weight in the range of from about 100 g/mol to about 1,000 g/mol.

51. (canceled)

52. A method of treating a disorder selected from the group consisting of meibomian gland dysfunction and dry eye disease, consisting of topically administering to the eyelid margin of a patient in need thereof a therapeutically effective amount of a pharmaceutical composition to treat the disorder, wherein the pharmaceutical composition consists of:
(a) mineral oil;
(b) a gelling agent; and
(c) optionally one or more pharmaceutically acceptable carriers and/or excipients.

53. The method of claim 52, wherein mineral oil is present in the pharmaceutical composition in an amount of at least 90% (w/w) of the pharmaceutical composition.

54. (canceled)

55. The method of claim 1, wherein an amount of from about 45 μL to about 55 μL of the pharmaceutical composition is topically administered to the eyelid margin of the patient.

56. (canceled)

57. (canceled)

58. (canceled)

59. (canceled)

60. The method of claim 1, wherein the pharmaceutical composition is administered twice per day.

61. (canceled)

62. The method claim 1, wherein the pharmaceutical composition is administered twice per day, wherein there is from about 8 hours to about 12 hours between administering a first dose of pharmaceutical composition and administering a second dose of pharmaceutical composition.

63-67. (canceled)

68. The method of claim 1, wherein after a duration of at least three months where the patient has received a dose of pharmaceutical composition each day, the patient has a vascular engorgement score of no greater than 1.5.

69. The method of claim 1, wherein the method produces a reduction in Eye Discomfort Visual Analog Score of at least 20 percent.

70. (canceled)

71. The method claim 1, wherein after a duration of at least three months where the patient has received a dose of pharmaceutical composition each day, the patient has an Eye Discomfort Visual Analog Score of no greater than 40.

72-74. (canceled)
